(19) United States
(12) Patent Application Publication
(10) Pub. No.: US 2026/0237528 A1
(43) Pub. Date: Aug. 13, 2026

(54) DRUG COMBINATIONS SCREENING

(71) Applicant: Hoffmann-La Roche Inc., Little Falls, NJ (US)

(72) Inventors: Mike BAKER, Muttenz (CH); Naghmeh GHAZALEH, Bottmingen (CH); Helen Katharina HAYWARD-KOENNECKE, Zollikerberg (CH); James OVERELL, Therwil (CH); Ali SAADAT VARNOSFADERANII, Basel (CH); Gian-Andrea THANEI, Turgi (CH)

(21) Appl. No.: 19/638,917

(22) Filed: Apr. 3, 2026

Related U.S. Application Data
(63) Continuation of application No. PCT/EP2024/078058, filed on Oct. 4, 2024.

(30) Foreign Application Priority Data
Oct. 6, 2023 (EP) 23202094.1

(51) Int. Cl. G16H 70/40; G06N 20/00

(52) U.S. Cl. CPC G16H 70/40; G06N 20/00

(57) ABSTRACT

Methods of screening drug combinations are described. The methods comprise obtaining, for a first drug and a second drug, the values of a plurality of features associated with the drug combination; and predicting, using the values of said plurality of features, the effect of the drug combination using a machine learning model that has been trained to predict whether a drug combination is likely synergistic. The plurality of features comprises at least one feature that characterises the similarity between one or more indications or diseases associated with one or more targets of the first drug and one or more indications or diseases associated with one or more targets of the second drug. Related methods, products and systems are also described.

### CROSS REFERENCE TO RELATED APPLICATIONS

This application is a continuation of International Application No. PCT/EP2024/078058, filed internationally on Oct. 4, 2024, which claims priority to European Patent Application No. 23202094.1, filed on Oct. 6, 2023, the disclosures of which are herein incorporated by reference in their entirety.

### FIELD OF THE PRESENT DISCLOSURE

The present disclosure relates to methods for predicting the effect of drug combinations, and in particular to the use of machine learning to predict the effect of treatment with pairs of candidate drugs for a particular condition from characteristics of the individual drugs in the pair. Related methods, systems and products are described.

### BACKGROUND

Despite advancements, effective treatments for multiple sclerosis (MS) progression and disability remain limited. Combining drugs has shown promise in areas like oncology and diabetes. However, screening new drug combinations is costly, time-consuming, and often infeasible to do at scale even remotely close to exhaustive even when limiting to approved therapies. For diseases like MS where known disease modifying therapies are scarce, the real potential is in exploring combinations with drugs not already indicated for the treatment of the disease, which is a space of combinations that is simply to vast to explore experimentally.

Computational methods for predicting the effect of drug combinations exist, and are reviewed in Paltun et al. 2021. These include machine learning based methods, amongst others, including methods that aim to identify new synergistic combinations (“drug synergy classification” methods). Work on previous such methods have indicated that drug target proteins, anatomical therapeutic chemical (ATC) codes and drug indications are informative in making such predictions (Paltun et al. 2021). ATC drug code information in particular has been identified to be the most useful information for such predictions. However, even with such information, the predictions of new combinations, especially involving drugs with no known drug combination, was found to be difficult.

For example, Ji et al. (2019) proposed a five-feature XGBoost model for classifying drug combinations as synergistic or antagonistic. The model uses: a disease intersection degree score calculated as the proportion of the indications of the two drugs that are identical, an adverse reaction intersection degree score calculated as the Jaccard similarity between the adverse drug reactions recorded for the two drugs, a biological process similarity score measured as a semantic similarity between gene ontology annotations for the targets, enzymes and transporters of the two drugs, a similarity of mode of action score obtained by integrating drug-target interaction information (i.e. promotive/inhibitory interaction) and a directed and signed protein interaction network (i.e. interactions between proteins indicated as promotive or inhibitory), and a separation score which quantifies the network-based separation of two drugs on a protein-protein interaction networks. Chemical structure similarity and ATC similarity were also tested but excluded from the final model after feature selection.

Existing methods still have prediction accuracies that can be improved upon, and rely heavily on predictive features such as ATC codes that are not available for new drug candidate compounds, further limiting their prediction accuracy for any combination involving such a drug.

Therefore, a need exists for improved systems and methods for predicting the effect of drug combinations.

### SUMMARY

There present inventors set out to build a predictive model to find a ‘high potential drug combination’ for improving the treatment of MS using machine learning. In this process, they developed a new machine learning approach to predict the effect of drug combinations which makes use of new predictive features that were found to contribute to high prediction accuracy. In particular, the inventors defined a new type of predictive feature for characterising drug combinations, which are based on similarity of disease related annotations of the drug targets. Existing methods make extensive use of disease indication information associated with the drug themselves, and in particular ATC codes. However, such disease indication information is only available for drugs that have been assigned an ATC code. ATC codes are assigned by the WHO on application by the drug owner, and requires a market authorisation application to be ready for submission In at least one country. Therefore, drugs that are still in clinical trial typically do not have an ATC code. By contrast, the present inventors proposed new predictive features that use quantify similarity of disease indication and/or association information by measuring (a) the overlapping Medical Subject Headings that exist for each drug target in the database, and/or (b) the similarity between disease ontology associated with the drug targets. These effectively quantify target disease similarity based on information about the drug targets rather than the drug themselves. The use of medical subject headings information in particular was found to be even more informative than ATC-based metrics, which were previously believed to be the most informative features for such models.

The present inventors showed that the newly developed models have extremely high prediction performance (specificity 94%, sensitivity 70%, accuracy 90%) on hold out test data comprising approved drugs. They further showed that the model was applicable to disease contexts that are poorly captured by existing models because of a sparsity of drugs for which information such as ATC codes is available, as the most informative features of the present model were newly proposed features that do not rely on this information, and for which the missing data problem was greatly reduced.

The newly developed approaches and models are applicable to any disease setting and any type of drugs, and were tested validated on a dataset comprising multiple thousands of drug combinations tested in clinical trials.

According to a first aspect of the disclosure, there is provided method for predicting the effect of a drug combination, the method including the steps of: obtaining, for a first drug and a second drug, the values of a plurality of features associated with the drug combination; and predicting, using the values of said plurality of features, the effect of the drug combination using a machine learning model that has been trained to predict whether a drug combination is likely synergistic, using training data comprising for each of a plurality of training drug combinations: (i) the values of said plurality of features and (ii) an indication of whether the drug combination is synergistic; wherein the plurality of features comprises at least one feature that characterises the similarity between one or more indications or diseases associated with one or more targets of the first drug and one or more indications or diseases associated with one or more targets of the second drug.

The method of the first aspect may have any one or any combination of the following optional features.

The machine learning model may have been trained to classify drug combinations between a plurality of categories comprising a synergistic category and a non-synergistic category, and predicting the effect of the drug combination may comprise determining whether the drug combination is likely to be in the synergistic category. The training data may therefore include a label indicative of a category of the plurality of categories. In other words, the indication of whether the drug combination is synergistic may be in the form of a label indicative of a category of the plurality of categories.

The plurality of features may comprise at least one feature that characterises the similarity between one or more indications associated with one or more targets of the first drug and one or more indications associated with one or more targets of the second drug, wherein indications are provided as Medical Subject Headings terms associated with the one or more targets of the drugs. The plurality of features may comprise at least one feature that characterises the similarity between one or more diseases associated with one or more targets of the first drug and one or more diseases associated with one or more targets of the second drug, wherein diseases are provided as disease ontology terms associated with the one or more targets of the drugs.

The similarity between one or more indications or diseases associated with one or more targets of the first drug and one or more indications or diseases associated with one or more targets of the second drug may be obtained as a semantic similarity metric between indication or disease terms associated with the one or more targets of the first drug and indication or disease terms associated with the one or more targets of the second drug, or a summarised metric that aggregates a plurality of such semantic similarity metrics each corresponding to a pair of indication or disease terms comprising an indication or disease term associated with a target of the first drug and an indication or disease term associated with a target of the second drug.

The plurality of features may comprise: one or more drug similarity features, one or more target similarity features, and one or more indication similarity features. The one or more drug similarity features may be selected from: a side effect similarity, a mode of action similarity and a molecular structure similarity. The one or more target similarity features may be selected from: a pathway similarity, a disease association similarity that characterises the similarity between one or more diseases associated with one or more targets of the first drug and one or more indications or diseases associated with one or more targets of the second drug, and a metric of closeness of the targets of the first and second drugs on a protein-protein interaction network. The one or more indication similarity features may be selected from: an indication similarity metrics based on the target(s) of the drugs that characterises the similarity between one or more indications associated with one or more targets of the first drug and one or more indications associated with one or more targets of the second drug, and an indication similarity metric based on the drugs that characterises the similarity between indication annotations associated with the first and second drugs.

The indication annotations may be provided as anatomical therapeutic chemical (ATC) codes. The indication similarity metric based on the drugs may be selected from: a similarity metric that quantifies the similarity between multiple levels of the ATC codes for the first and second drugs, and a similarity metric that compares the first level of the ATC codes for the first and second drugs. The one or more indication similarity features may comprise a similarity metric that quantifies the similarity between multiple (optionally all) levels of the ATC codes for the first and second drugs, and one or more similarity metrics that compares the first level of the ATC codes for the first and second drugs. A similarity metric that compares the first level of the ATC codes for the first and second drugs may be a binary feature that indicates whether the first and second drugs are associated with ATC codes in the same first level category, or in a particular pair of ATC categories. The plurality of features may comprise a set of binary features that indicate whether the first and second drugs are in each of a plurality of pairs of first level ATC categories. The plurality of pairs of first level ATC categories may include LL, NN, JJ and CC.

The machine learning model may be selected from: decision trees and variants thereof including regularised and/or gradient boosted decision trees and random forest models, regularised discriminant analysis, logistic regression models, artificial neural networks, naïve Bayes classifiers, support vector machines and multivariate adaptive regression splines. The machine learning model may be a non-linear model. The machine learning model may be an ensemble model. The machine learning model may be a tree-based model. In embodiments, the machine learning model is a gradient boosted tree model.

The plurality of features may comprise: (i) at least one feature that characterises the similarity between one or more indications associated with one or more targets of the first drug and one or more indications associated with one or more targets of the second drug, wherein indications are provided as Medical Subject Headings terms associated with the one or more targets of the drugs; (ii) a side effect similarity, (iii) a mode of action similarity, (iv) a molecular structure similarity, (v) a pathway similarity, (vi) a disease association similarity that characterises the similarity between one or more diseases associated with one or more targets of the first drug and one or more indications or diseases associated with one or more targets of the second drug, and a metric of closeness of the targets of the first and second drugs on a protein-protein interaction network, and (vii) an indication similarity metric based on the drugs that characterises the similarity between indication annotations associated with the first and second drugs, wherein the indication annotations are provided as anatomical therapeutic chemical (ATC) codes and wherein the indication similarity metric based on the drugs comprises a similarity metric that quantifies the similarity between multiple levels of the ATC codes for the first and second drugs. The indication similarity metric based on the drugs may further comprise a similarity metric that compares the first level of the ATC codes for the first and second drugs, wherein the similarity metric that compares the first level of the ATC codes for the first and second drugs is a set of binary features that indicate whether the first and second drugs are in each of a plurality of pairs of first level ATC categories, optionally wherein the plurality of pairs of first level ATC categories include LL, NN, and optionally one or more of JJ, CC, JS, DR, DS, CJ, AA, AM, AN, and BN.

The machine learning model may have been trained using a training data set comprising at least 1000, 2000 or 3000 unique drug combinations. The trained machine learning model may have a specificity of at least 90% and sensitivity of 70% in predicting synergistic combinations when evaluated on unseen data. The method may further comprise prior to providing the values said plurality of features as inputs to said machine learning model, applying one or more steps selected from: missing values imputation, filtering, standardisation and normalisation.

Embodiments of any aspects may comprise providing the results of any step to a user, for example through a user interface.

According to a second aspect, there is provided a method of providing a tool for predicting the effect of a drug combination, the method comprising: obtaining, for each of a plurality of training drug combinations comprising a first drug and a second drug: (i) the values of a plurality of features associated with the drug combination; and (ii) information indicative of whether the drug combination is synergistic; and training a machine learning model to predict whether a drug combination is likely synergistic, using said training data, wherein the machine learning model is trained to take as input the values of said plurality of features for a drug combination and to produce as output an indication of whether the rug combination is likely to be synergistic; wherein the plurality of features comprise at least one feature that characterises the similarity between one or more indications or diseases associated with one or more targets of the first drug and one or more indications or diseases associated with one or more targets of the second drug.

The methods according to the present aspect may have any one or more of the features specified in relation to the first aspect.

According to a third aspect, there is provided a method of identifying a synergistic drug combination, the method comprising: obtaining a plurality of candidate drug combinations; predicting the effect of each of the plurality of candidate drug combinations using the method of any embodiment of the first aspect; and selecting a subset of the plurality of candidate drug combinations for experimental testing using the results of the predicting. The method may further comprise testing the selected subset of the plurality of candidate drug combinations using one or more in vitro assays.

The candidate drug combinations may each comprise a drug that is approved or in clinical or preclinical trial for the treatment of a particular disease or disorder, such as e.g. MS.

According to a fourth aspect, there is provided a system comprising: at least one processor; and at least one non-transitory computer readable medium containing instructions that, when executed by the at least one processor, cause the at least one processor to perform the method of any embodiment of any of the first, second or third aspect, or any method described herein.

According to a further aspect, there is provided a non-transitory computer readable medium comprising instructions that, when executed by at least one processor, cause the at least one processor to perform the method of any embodiment of any of the first, second or third aspect, or any method described herein.

According to a further aspect, there is provided a computer program comprising code which, when the code is executed on a computer, causes the computer to perform the method of any embodiment of any of the first, second or third aspect, or any method described herein.

### BRIEF DESCRIPTION OF THE DRAWINGS

Embodiments of the present disclosure will now be described by way of example with reference to the accompanying drawings in which:

FIG. 1 is a flowchart illustrating a method for predicting the effect of drug combinations according to a general embodiment of the disclosure;

FIG. 2 is a flowchart illustrating a method for providing a tool for predicting the effect of drug combinations according to an embodiment of the disclosure;

FIG. 3 illustrates schematically an exemplary system according to the disclosure;

FIG. 4 illustrates schematically a process of predicting the effect of drug combinations implemented examples of the disclosure.

FIGS. 5A-5B illustrate schematically the concept of a metric of closeness of drug targets in a protein-protein interaction (PPI) network. FIG. 5A. Example for two overlapping drugs. FIG. 5B. Example for two separated drugs.

FIG. 6 illustrates schematically a data structure used in examples of the disclosure.

FIG. 7 shows missing data in predictive features for training data used in examples of the disclosure. Rows are pairs of drugs in a training dataset, and columns are predictive features for the pairs of drugs. Percentages in the column labels are the % missing data for the specific feature. Se_sim-side effect similarity; kegg_sim=pathway similarity (keg pathway database); atc_sim=ATC code similarity; mesh_sim=MESH similarity; ppi_mean_dist=mean distance between drug target clusters in PPI network; do_sym=disease ontology similarity; moa_sim=mode of action similarity; smiles_sim=molecular structure similarity.

FIGS. 8A-8B show the results of a feature selection process used in examples of the disclosure. FIG. 8A. F1 score with progressively smaller sets of predictive features. FIG. 8B. Tolerance (loss in F1 performance compared to the best model, %). Triangle indicates the selected model (8 features). Solid dot indicates the best model (11 features). A tolerance of 5% was applied for model selection (dashed horizontal line).

FIGS. 9A-9C show the results of an investigation of predictive feature importance (1-AUC for 10 random permutations of the respective features) in a model according to the disclosure. FIG. 9A. Individual predictive features, ATC aggregated (i.e. all ATC related features considered together, including ATC code similarity and pairwise possibility of combination of 14 different first level value of the ATC code). FIG. 9B. Individual predictive features, selected ATC binary categories shown. FIG. 9C. Importance aggregated by categories of predictive features.

FIGS. 10A-10B show the correlation between predictive features in an example of the disclosure. FIG. 10A. Correlation between 8 features used in a model after feature selection (atc_sim shows only the full code similarity). Feature legends are as in FIG. 7. FIG. 10B. Correlation between all features considered, except for the ATC first level binary categories for which only the most important categories are shown. Feature legends are as in FIG. 7 except that ATC first level binary categories with high importance are each indicated as “atc_xx” where “xx” refer to the first left of the ATC code of each of the respective drugs (e.g. “atc_Il” is the value of a binary variable that is “1” if the two drugs are in ATC first level category L, and “0” otherwise). On both plots, the colour intensity and size of the dots indicate the magnitude of the correlation.

FIGS. 11A-11B show the distribution of predictive features values, for each of a plurality of predictive features, separated by antagonistic examples and synergistic examples. FIG. 11A. Individual predictive features, ATC aggregated (i.e. all ATC related features considered together, including ATC code similarity and pairwise possibility of combination of 14 different first level value of the ATC code). FIG. 11B. Individual predictive features, selected ATC binary categories shown. MESH=mesh similarity, molecular structure=molecular structure similarity, network separation=PPI closeness, MOA=mode of action similarity, pathway=pathway similarity (kegg), ATC=aggregated ATC similarity, Disease ontology=disease ontology similarity, side effect=side effect similarity.

FIGS. 12A-12D show results of a comparison of different model architectures and data preprocessing pipelines. FIG. 12A. F1 score (top) and balanced accuracies obtained on a hold out test data set using different model architectures and data preprocessing pipelines. FIG. 12B. Posterior distributions of mean balanced accuracies obtained on a hold out test data set using different model architectures and data preprocessing pipelines. FIG. 12C. Comparison of posterior for mean balance accuracy on hold out test data for random forest models on data processed by imputation only vs XGBoost models on data processed by imputation and normalisation. Boost_tree=gradient boosted trees (XGBoost), decision_tree=decision tree ensemble, discrim_regularized=regularised discriminant analysis, logistic_reg=logistic regression, mars=multivariate adaptive regression spline, mlp=multilayer perceptron, rand_forest=random forest, svm_rbf=support vector machine with radial basis function kernel, rec_impute=data processed with imputation only, rec_impute_norm=data processed with imputation followed by standardisation and normalisation, rec_impute_norm_pca=data processed with imputation followed by standardisation and normalisation followed by dimensionality reduction by principal component analysis (PCA), rec_impute_norm_pls=data processed with imputation followed by standardisation and normalisation followed by dimensionality reduction by partial least square regression (PLS). FIG. 12D. Practical equivalence of workflow sets: this proportion of models obtained on resampled data (from cross-validation) that have an effect size of at most 2% (loss in mean balanced accuracy compared to best model). This shows that the XGboost model performs best on both raw and normalised data, and the random forest model is about 68% practically equivalent to the XGboost model.

FIGS. 13A-13B shows the results of threshold selection and optimisation processes used to obtain a model of the disclosure (XGboost model). FIG. 13A. F1 score, precision (increasing data series) and recall (decreasing data series) obtained by XGBoost models as the threshold for calling predictions as “positive” (synergistic) increases, for the training data. FIG. 13B. Balanced accuracy, sensitivity (decreasing data series) and specificity (increasing data series) obtained by XGBoost models as the F1 threshold increases (same models as on FIG. 13B, but evaluating on different performance metrics). The dashed line shows the threshold selected based on F1 score.

FIGS. 14A-14B shows prediction performance of a model of the disclosure (XGBoost) evaluated on a hold out test dataset. FIG. 14A. confusion matrix obtained by evaluating the trained XGBoost model on the hold out test data. FIG. 14B. Receiver operator characteristic (ROC) curve obtained by applying different predicted probability thresholds to the model results.

FIG. 15 is a calibration curve showing the observed numbers of synergistic pairs (event rate) for each of a plurality of bins of predicted probabilities from the XGboost model (x axis indicating the midpoint of the bins). The 0-1 probability range was divided into 10 equal size bins [0,0.1) [0.1,0.2) . . . [0.9,1], with the indicated mid points, i.e. 0.05, 0.15, . . . , 0.95. The proportion of synergistic pairs amongst the pairs predicted to have a probability falling in each bin is plotted (solid point), together with the range of predicted probabilities for these observations.

FIG. 16 shows partial dependence profiles for pairs of predictive features. Mesh_sim=MESH similarity, ATC_sim=ATC similarity (complete ATC code similarity), do_sim=disease ontology similarity, smiles_sim=structural similarity, kegg_sim=pathway similarity, se_sim=side effects similarity, ppi_separation=closeness of drug targets on PPI network, moa_sim=mode of action similarity. Partial dependence profiles show how the expected value of model prediction (here synergy probability, on the y-axis) behave as a function of a selected explanatory variable (indicated on the x axis). Each grey line corresponds to a single drug combination, and the bold blue line is the average of all the grey lines. The value of a PD profile at value z of an explanatory variable is the empirically estimated expected value of the model predictions when the explanatory variable is fixed at value z over the joint distribution of all other explanatory variables.

FIG. 17 shows the missing values for pairs of drugs each comprising a MS known disease modifying therapy. Each row is a drug combination, and black indicates a missing value. Mesh_sim=MESH similarity, ATC_sim=ATC similarity, do_sim=disease ontology similarity, smiles_sim=structural similarity, kegg_sim=pathway similarity, se_sim=side effects similarity, ppi_separation=closeness of drug targets on PPI network, moa_sim=mode of action similarity. The percentage missing data is indicated for each feature separately on the x axis labels.

FIG. 18 shows a histogram of the distribution of the number of missing features per combination in the data on FIGS. 17-18 (i.e. each bar represents the number of combinations in the MS dataset that had the stated number of missing features). There were 33,313 combinations for which the maximum number of missing features was 2 (i.e. 0, 1 or 2 missing features).

FIG. 19 is a histogram showing predicted probability of synergy for each of the 33,313 combinations highlighted on FIG. 18, together with the cutoff on the predicted probability (dashed vertical line) and the % of combinations that were predicted as synergistic. The top predicted pairs are listed in the inset panel together with the MS disease modifying therapy (DMT) in the first column, the additional therapy in the second column and the probability predicted by the model in the third column.

FIG. 20 shows the feature importance for one of the top predicted combinations in the data on FIG. 19. Each row is a feature, the y-axis label indicates the feature and the value of the feature for the particular drug combination. The shaded values are the average contribution (Shapley additive explanations) of the feature to the probability predicted by the model for the pair, and the boxplots show the distribution of contributions obtained across multiple possible orderings used to compute the Shapley attributions.

Where the figures laid out herein illustrate embodiments of the present invention, these should not be construed as limiting to the scope of the invention. Where appropriate, like reference numerals will be used in different figures to relate to the same structural features of the illustrated embodiments.

### DETAILED DESCRIPTION

Specific embodiments of the invention will be described below with reference to the figures.

The present disclosure describes methods for predicting the effect of a drug combination using a machine learning model that takes as input values for a plurality of predictive features about the drug combination.

In the context of the present disclosure, a “drug” refers to any active substance for use in medicine. A rug may also be referred to as a “therapeutic”. This includes in particular small molecules and large molecules (e.g. antibodies). A drug may advantageously be any active substance that modulates the activity of one or more targets. Targets may be genes or expression products thereof (e.g. proteins). In other words, a drug may be any active substance that can be associated with one or more targets, where the drug modifies the activity of the target(s). The term is not limited in terms of how the activity of the target is modulated, whether the modulation is direct or indirect, or whether the modulation is positive or negative. Further, drugs without known targets may also be included provided that enough predictive features about the drug can be determined. As explained below, predictive features may include features that are associated with the drug target, features that are associated with the drug indication and features that are associated with the drug structure. Therefore, predictive features may still be obtainable for a drug without known targets, and such a drug may still be used for training and/or for prediction using machine learning as described herein when more than half or more than two thirds of the predictive features used can be determined (e.g. 5 or more out of 8 or 9 predictive features, preferably 6 or more of 8 or 9 predictive features). For the purpose of training a machine learning model as described herein, it is advantageous to use a training dataset comprising a majority of drugs combinations (e.g. at least 50%, at least 60%, at least 70%, at least 80% or at least 90%) where both drugs have at least one known target.

A drug combination refers to the administration of a pair of drugs to a subject. Thus, predicting the effect of a drug combination refers to predicting the therapeutic effect that the drug combination would have if administered to a subject. This may comprise classifying the drug combination between a plurality of classes including one or more synergistic classes and one or more non-synergistic classes as will be described further below. It may be particularly advantageous to simply distinguish between synergistic and non-synergistic drug combinations. Indeed, this produces a simple classification that is easier to fit and easier to assign labels for training purposes. This further reflects clinical reality where drug combinations are more likely to be considered promising if they have a synergistic effect because this advantageously balances the additional risks and side effects associated with administering a pair of drugs rather than a single drug. The present disclosure relates primarily to methods of predicting the effect that a drug combination would have if administered to a subject, but do not typically comprise any step of administering a drug or drug combination to a subject. Nevertheless, the prediction is not limited to any administration mode, or to concurrent or subsequent administration of the drugs. Indeed, the predictions are based purely on characteristics of the drug that are inherent to their function and structure. It is beyond the scope of the present disclosure to determine the formulation and therapeutic regimen to be used to administer a drug combination that has been selected as likely synergistic using the methods described herein, and experimentally validated.

The term “machine learning model” refers to a mathematical model that has been trained to predict one or more output values (predicted variables) based on input data (predictive variables, also referred to as values of predictive features). Training refers to the process of learning, using training data, the parameters of the mathematical model that result in a model that can predict outputs values that satisfy an optimality criterion or criteria. In the case of supervised learning, training typically refers to the process of learning, using training data, the parameters of the mathematical model that result in a model that can predict output values that with minimal error compared to comparative (known) values associated with the training data (where these comparative values are commonly referred to as “labels” or “ground truth”). The are two major types of supervised learning models: classification models and regression models. Classification models aim to classify observations between a plurality of categories. They are typically suitable when the output to be predicted is a categorical label in the training data (e.g. drug combination is synergistic or not synergistic). Regression models aim to predict the value of a continuous variable associated with observations (e.g. drug combination has a continuous quantified effect on an outcome variable). They are typically suitable when the output to be predicted is a continuous value in the training data. A classification model may provide as output a classification label and/or one or more probabilities of an observation belonging to respective one or more classes. A binary classification model may provide as output a single probability indicating the probability that the observation belongs to a positive class (e.g. synergistic drug combinations). A predetermined threshold may be applied to the one or more probabilities to assign a class label. The threshold may be determined based on desired characteristics of the classification, such as e.g. a desired level of specificity, sensitivity or any accuracy performance that combines aspects of specificity (precision) and sensitivity (recall), such as accuracy and F1 score or balanced versions thereof that take into account the proportions of observations in the training data in each of the classes.

Embodiments of the present disclosure make use of classification models that are trained using training data comprising pairs of drugs and associated labels identifying the drug combination as belonging to one of a plurality of categories including at least a “synergistic” category. The plurality of categories may comprise a first category of synergistic drug combinations and a second category of non-synergistic drug combinations. When a binary classification model is used (i.e. the plurality of categories consist of said synergistic and non-synergistic categories), the latter may also be referred to as “antagonistic” drug combinations even though they may strictly speaking not be antagonistic (i.e. their combined effect may not be worse than their individual effect, but their combined effect is not better than the sum of their individual effects). A synergistic drug combination refers to a drug combination where the effect of the combination on at least one clinical endpoint is better than the sum of the effects of each of the drugs individually. The term “better” refers to an improvement of the endpoint, which may be a reduction or an increase depending on the situation (i.e. depending on whether the endpoint is something to be reduced by treatment—e.g. presence and/or extent of lesions—or increased by treatment—e.g. a motor function score). A non-synergistic drug combination refers to a drug combination where the effect of the combination on at least one clinical endpoint is not better than the sum of the effects of each of the drugs individually, and/or the side-effects of the combination are as bad or worse than the sum of the side-effects of each of the drugs individually. Additional and/or subsets of these categories may be used. For example, the plurality of categories may comprise a first category of synergistic drug combinations, a second category antagonistic drug combinations, and a third category of additive drug combinations. In such embodiments, an antagonistic drug combination may refer to a drug combination where the effect of the combination on at least one clinical endpoint is worse than the sum of the effects of each of the drugs individually and/or the side-effects of the combination are worse than the sum of the side-effects of each of the drugs individually. An additive drug combination may refer to a drug combination where the effect of the combination on at least one clinical endpoint is equal to the sum of the effects of each of the drugs individually and the side-effects of the drug combination are not worse than the sum of the side-effects of each of the drugs individually. Training data comprising pairs of drugs and associated labels identifying the drug combination as belonging to one of a plurality of categories including at least a “synergistic” category is available in multiple online resources, such as e.g. the DrugComb database (Liu et al. 2020; drugcombdb.denglab.org/). Synergistic drug combinations are recorded in the FDA's “Approved Drug Products with Therapeutic Equivalence Evaluations” publication, commonly known as the “orange book” (FDA, 2023). Therefore, synergistic drug combinations for the purpose of training machine learning models as described herein may be obtained from the FDA orange book. Such data can be downloaded from the DrugComb database (Liu et al. 2020). Non-synergistic drug combinations data is available in DrugBank (Wishart et al. 2017) and can be downloaded from the DrugComb database.

The term “machine learning algorithm” or “machine learning method” refers to an algorithm or method that trains and/or deploys a machine learning model. The machine learning models of the present disclosure are trained by supervised learning. An optimality criterion may be the minimisation of a loss function that quantifies the model prediction error based on the observed (ground truth) and predicted values of the predicted variables. Suitable loss functions for use in training machine learning models are known in the art and include the mean squared error, and the mean absolute error. Any of these can be used according to the present disclosure. The mean squared error (MSE) can be expressed as L(·)=MSE(xᵢ, \hat{x}ᵢ)=(xᵢ−\hat{x}ᵢ)² where xᵢ and \hat{x}ᵢ are the predicted and observed (ground truth) values for a data instance (in the present case, a candidate drug combination in the training data), respectively. The mean absolute error (MAE) can be expressed as: L(·)=MAE (xᵢ, x̅ᵢ)=|xᵢ−x̅ᵢ| where xᵢ and x̅ᵢ are the predicted and observed (ground truth) values for a data instance (in the present case, a candidate drug combination in the training data), respectively. The MAE is believed to be more robust to outlier observations than the MSE. The MAE may also be referred to as “L1 loss function”. However, MSE remains a very commonly used loss functions especially when a strong effect from outliers is not expected, as it can make optimization problems simpler to solve. Regularised loss functions are functions that include a loss function as described above, and one or more terms penalizing model complexity in order to reduce the risk of overfitting. Overfitting is a phenomenon that occurs where a machine learning model is trained to very closely reproduce the features of a training data set, resulting in poorer performance on other datasets that do not have the same characteristics (i.e. poor generalizability). L1 regularisation (also known as “Lasso” in the context of regression) add a regularization term to the loss function that penalizes models based on the sum of absolute value of the coefficients of the model. L2 regularisation (also known as “Ridge” in the context of regression) add a regularization term to the loss function that penalizes models based on the sum of squared value of the coefficients of the model. L1 regularisation can be used as a feature selection method as it minimizes the coefficients associated with less informative predictive features. In embodiments, the machine learning model is a regularized model. In embodiments, the machine learning model is a regularized tree-based model. In embodiments, the machine learning model is a regularized gradient boosted decision tree model. Examples of such models are available in the XGBoost software library (xgboost.ai/). Such models may be referred to as “XGBoost” models, although any other implementation of regularized gradient boosted models may equally be used.

A machine learning model as described herein may be selected from: decision trees and variants thereof including regularised and/or gradient boosted decision trees and random forest models, regularised discriminant analysis, logistic regression models, artificial neural networks (ANNs) including multilayer perceptrons (with linear or non-linear activation functions) and deep learning models (e.g. long short-term memory networks (LSTMs), Recurrent neural networks (RNNs), Generative Adversarial Networks (GANs), etc), naïve Bayes classifiers, support vector machines (SVM, using linear or non-linear kernels such as radial basis function) and multivariate adaptive regression splines (MARS). The present inventors have found it to be particularly beneficial to use non-linear models, such as decision trees and variants thereof (including in particular random forests and gradient boosted trees), SVM with a non-linear kernel, and ANNs (e.g. multilayer perceptrons) with non-linear activation functions. Relatively simple ANNs such as MLPs were preferred in this particularly work because more complex deep learning models such as LSTMs, RNNs and GANs typically have more trainable parameters and therefore perform best when trained with large amounts of training data.

In embodiments, a machine learning model comprises an ensemble of models whose predictions are combined. Alternatively, a machine learning model may comprise a single model. Random forest models and gradient boosted tree models (such as XGBoost) are ensemble models. Ensemble versions of any models can be constructed. Ensemble models are expected to result in better prediction performance than single models and are therefore preferred in the context of the methods described herein. For example, the machine learning model may be a random forest classifier or a gradient boosted decision tree model. A random forest classifier is a model that comprises an ensemble of decision trees and outputs a class that is the average prediction of the individual trees. Decision trees perform recursive partitioning of a feature space until each leaf (final partition sets) is associated with a single value of the target. Gradient boosting is a machine learning method that forms an ensemble of weak prediction models (e.g. decision trees) from which a combined strong prediction is obtained. The algorithm iteratively adds new weak predictors to improve the prediction obtained by combining the outputs of the weak predictors. By contrast, random forest iteratively trains a set number of trees using random subsets of the training data.

The methods described herein predict the effect of a drug combination based on the value of one or more predictive features. Predictive features (also referred to herein simply as “features”) are characteristics of a drug combination that can be quantified and that characterize properties of the drug combination other than the effect of the drug combination. The effect of the drug combination is a predicted feature of a machine learning model according to the disclosure. The predictive features according to the present disclosure may include one or more features that are indicative of the similarity between the indications of the drugs in the combination (indication similarity features). The predictive features according to the present disclosure may include one or more features that are indicative of the similarity between the targets of the drugs in the combination (target similarity features). The predictive features according to the present disclosure may include one or more features that are indicative of the similarity between the drugs in the combination (drug similarity features).

Indication similarity may include one or more indication similarity metrics based on the target(s) of the drugs, and/or one or more indication similarity metrics based on the drug. In embodiments, the one or more predictive features used by the machine learning model include at least one indication similarity metric based on the target(s) of the drug. An indication similarity metric based on the targets of the drug may be a similarity metric that quantifies the similarity between medical annotations associated with the targets of the drugs. For example, Medical Subject Headings (MeSH or MESH-see www.nlm.nih.gov/mesh/meshhome.html) annotations can be associated with genes/proteins as described in Yu 2018. In particular, genes can be associated with MESH terms by text mining, for example by pairing entries from the Online Mendelian Inheritance in Man (OMIM) database with MESH terms as described in Nakazato et al. 2009 (providing gene-level MESH annotations available at gendoo.dbcls.jp), and/or by use of annotations between Entrez gene identifiers and Pubmed articles in the gene2pubmed data (Maglott et al. 2007; available in the “Related articles in PubMed” section of any entry of the Entrez gene database www.ncbi.nlm.nih.gov/gene/as well as a bulk download on the NGBI website), where PubMed articles are associated with MESH terms by manual curation (as the MESH vocabulary was developed for the primary purpose of classifying PubMed content). Sequence similarity tools may additionally be used to provide annotations for genes that lack an annotation, for example reciprocal blast hits (RBBH) may be used to assign the annotation of a most similar gene in a different genome. This may in particular be used to use annotations from a gene in one species to an ortholog gene in another species. The annotations used in the present examples make use of both annotations obtained by pairing entries from OMIM with MESH terms and annotations obtained from the gene2pubmed data. Thus, in embodiments, indication similarity metric based on the targets of the drug may be a similarity metric that quantifies the similarity between medical annotations associated with the targets of the drugs provided as MESH term annotations associated with the gene identifiers of the targets, where the MESH term annotations are annotations obtained by pairing entries from OMIM with MESH terms and/or annotations obtained from the gene2pubmed data.

An indication similarity metric based on the drug may be a similarity metric that quantifies the similarity between indication annotations directly associated with the drug. For example, ATC codes are indication annotations associated with drugs. A similarity between ATC codes may be a metric that quantifies the similarity between complete ATC codes (or at least multiple levels of the ATC codes) or between the first level of the ATC classification. A similarity between complete ATC code may be obtained using any similarity metric that is applicable to compare discrete sets (e.g. Jaccard similarity, as described below) or any similarity metric that is applicable to compared discrete sets organised as ontologies (e.g. Wang, Resnik, Relevance, Jiang and Lin methods, further described below). Note that each drug is assigned a unique ATC code and therefore there is no need for aggregating similarity scores for multiple annotations when determining a similarity between complete ATC codes. In other words, each drug is associated with a single code, which completely defines the position of the drug in the ATC ontology. A similarity between complete ATC codes may be obtained using a similarity metric between discrete sets (e.g. Jaccard similarity) where the discrete set for a drug is obtained as the set of all possible subsets of the ATC code of the drug starting from the first level code and increasing by one character for each subset. For example, code L04AA36 may be represented by the set [L, L0, L04, L04A, L04AA, L04AA3, L04AA36]. A similarity between the first level of the ATC classification may be obtained as a binary feature that indicates whether the drugs that form part of a combination are associated with ATC codes in the same first level category, or in a particular pair of ATC categories. The first level categories of the ATC classification are listed in Table 1. For example, a pair of drugs that are both in first level class L may be associated with a similarity of “1”, whereas a pair of drugs that are in different first level classes (e.g. L and N, respectively) may be associated with a similarity of “0”. As another example, a pair of drugs that are both in first level class L (informative class pair) may be associated with a similarity of “1”, whereas a pair of drugs that are any other category or combination of categories may be associated with a similarity of “0”. Note that the informative class pair may be any first level class pair. For example, it may be relevant that the combination comprises a drug in class J and a drug in class S. The inventors have found that identical class pairs are more likely to be informative features, and therefore it is advantageous for such a similarity feature to indicate whether both drugs in a combination fall within the same specific level 1 class. Further, the present inventors have found specific pairs of classes to be particularly informative, such as LL, NN, JJ and CC. Each combination of first level classes that contributes to predictions may be referred to as an “informative class pair”. A similarity between the first level of the ATC classification may be obtained as a set of binary features (i.e. a binary vector) that indicates whether the drugs that form part of a combination are associated with each of a plurality of categories defined by a particular combination of first level classes. For example, each of the above informative class pairs (or any class pair that was found to be informative for a particular training dataset) may be represented. Alternatively, each possible class pair may be represented. Thus, a similarity between the first level of the ATC classification may be obtained as a set of binary features that include a “0” or a “1” at each of a plurality of positions of the binary vector that correspond to a particular class pair. Note that as drugs are assigned a single ATC code, such a binary vector can only contain a single “1”. The class pairs may comprise one or more of LL, NN, JJ, CC, JS, DR, DS, CJ, AA, AM, AN, BN. It is advantageous for the class pairs to include at least LL and NN.

Drug similarity features may include one or more features selected from: side effect similarity, mode of action similarity and molecular structure similarity.

Side effect similarity is a similarity that compares side-effects terms associated with the drugs in a drug combination. Side-effects are recorded using accepted terminology in databases such as the FDA Orange book. A list of side effects associated with a first drug can be compared with a list of side effects associated with a second drug using any similarity metric known in the art for comparing discrete sets. For example, a similarity between side effects associated with two drugs may be obtained as a Jaccard similarity. Note that in embodiments where side effects are specified according to a controlled vocabulary that is organised as an ontology, any similarity metric that is designed for ontologies may be used, as explained further below.

Mode of action similarity is a similarity that compares mode of action terms associated with the drugs in a drug combination. Mode of action terns are recorded using accepted terminology in databases such as DrugBank. A list of mode of action terms associated with a first drug can be compared with a list of mode of action terms associated with a second drug using any similarity metric known in the art for comparing discrete sets. For example, a similarity between mode of action terms associated with two drugs may be obtained as a Jaccard similarity. Note that in embodiments where the modes of action of drugs are specified according to a controlled vocabulary that is organised as an ontology, any similarity metric that is designed for ontologies may be used, as explained further below.

Molecular structure similarity is a similarity that compares the 2D and/or 3D structure of drugs in a drug combination. The 2D structure of a molecule may be represented as a fingerprint. This may be a numerical vector that indicates the present of particular substructures in a molecule (also known as a binary fingerprint). Any method known in the art to obtain a molecular fingerprint may be used, such as for example, the extended-connectivity fingerprint ECFP4 (described in Rogers and Hahn (2010)). Any similarity metrics known in the art for the purpose of comparing molecular fingerprints may be used, including e.g.: Tanimoto similarity (which is a Jaccard similarity), Dice similarity, Modified Tanimoto, Cosine similarity (see equation (0), Ochiai (ratio of the number of features common to both molecules to the square root of the product of the number of features in the two molecules), Kulczynski2 (ratio of (the number of features in common to both molecules multiplied by the average size of the total number of features present) divided by the product of the size of the total number of features present (i.e. ((A intersect B)*0.5(A+B))/(A*B))).

Target similarity features may include one or more of: pathway similarity, target protein similarity, disease association similarity, co-expression, and closeness on protein-protein interaction network (PPI). In embodiments, target similarity features include one or more or all of: pathway similarity, disease association similarity, and closeness on protein-protein interaction network (PPI). Note that indication similarity metrics that are based on the target(s) of the drug may also be seen as target similarity features, but were classified as indication similarity features because it was found that their information content was more similar to drug-associated indication similarity metrics.

Disease association similarity is a similarity that compares disease-related annotations associated with any known target of the drugs. Disease related annotations may be in the form of disease ontology terms. Disease ontology terms annotations for genes/proteins are available for example at the Disease and Gene Annotations database (DGA, dga.nubic.northwestern.edu; Peng et al. 2013). Any metric of similarity described below may be used. Preferably, the metric of similarity is one that is designed for ontologies (also referred to as semantic similarity in the present context), as described further below.

Pathway similarity is a similarity that compares pathway annotations associated with any known target of the drugs. The pathway annotations may be from any pathway annotation database, including Kegg (www.genome.jp/kegg/pathway.html), Reactome (reactome.org/), and Gene Ontology biological process annotations (see geneontology.org/). Reactome and Kegg provide pathway maps indicating genes involved in known biological pathways. The present inventors tested each of these sources of pathway information and found them to result in highly correlated predictive features, indicating that any of these could be used interchangeably. In embodiments, pathway annotations are obtained from Kegg. Each drug may be associated with one or more drug targets. Each drug target may be associated with one or more pathways. Therefore, each drug may be associated with a set of pathways, comprising all of the pathways associated with any of the targets of the drug. The pathways associated with a first drug can be compared to the pathways associated with the second drug by computing one or more pairwise similarities, one between each pair of pathways comprising a pathway associated with the first drug and a pathway associated with the second drug. Similarity between two pathways can be quantified using any metric applicable to discrete sets, by comparing the sets of genes involved in the two pathways. Similarity between two sets of pathways can be quantified by combining such similarities using any method known in the art for combining multiple pairwise similarities. Examples of such methods are provided further below.

Target protein similarity is a similarity metric that compares the targets of the two drugs in a pair. In embodiments, this is a score between 0 and 1 that quantifies the similarity between two lists, respectively including the targets of the first and second drug. Any similarity metric applicable to discrete sets, such as e.g. Jaccard similarity, may be used for this purpose.

A coexpression similarity is a similarity that compares expression profiles across a plurality of samples or conditions (including e.g. cell lines, organs, etc.) for each of a plurality of pairs of genes comprising a drug target of a first drug and a gene target of a second drug in a drug combination. For example, all pairwise comparisons between a target of the first drug and a target of the second drug may be performed (i.e. a similarity metric for each such comparison may be obtained), and the resulting one or more similarities (where the total number depends on the number of drug targets) may be combined using any method described herein. Similarities between expression profiles may be computed using any metric known in the art, such as e.g. Pearson correlation, Spearman rank correlation, cosine similarity, etc. The similarity metric may compare the average expression of all targets of drug 1 in each of a plurality of tissues, and the average expression of all targets of drug 2 in each of a plurality of tissues.

The closeness on a protein-protein interaction network is a similarity that compares subnetworks associated with drug targets of a first and second drug in a drug combination, where subnetworks are subsets of a protein-protein interaction (PPI) network. A PPI network is a network comprising nodes corresponding to genes/proteins, and edges corresponding to functional and/or physical interactions between the genes/proteins associated with the nodes that the edges connect. Any PPI network known in the art may be used, including any PPI network that combines information from multiple PPI networks. For example, a PPI network may be selected from STRING (e.g. STRING v11, available at string-db.org/and described in Szklarcyk et al. 2019), or any subset thereof including interactions from Kegg (Kanehisa et al. 2017), Reactome (Fabregat et al. 2016), BioCyc (Caspi et al. 2016) and/or pathways in Gene Ontology (The Gene Ontology, 2017). PPIs may be functional interactions. Functional interactions may include direct interactions in pathways and/or protein complex membership. A subnetwork associated with a drug may be obtained as the smallest subnetwork that includes all of the targets of the drug. This may include all of the nodes of a PPI that are targets of the drug, and all nodes that lie on the shortest path (or shortest paths, if there are multiple shortest paths) between nodes that are targets of the drug. Other criteria may be used to construct a subnetwork, such as e.g. including all nodes that correspond to drug targets and all nodes that are within a certain number of edges away from a drug target. A subnetwork associated with a drug may be the set of nodes that are targets of the drug. A closeness between such subnetworks may be obtained as a score that quantifies the distance and/or overlap between subnetworks. Where multiple distjoint subnetworks are present for at least one of the drugs, pairwise closeness metrics may be computed for all pairs of subnetworks comprising a subnetwork associated with the first drug and a subnetwork associated with the second drug of a drug combination. The resulting plurality of closeness metrics may be combined using any method known in the art for combining similarities, or using any summarised metric known in the art. A closeness between subnetworks that are sets of target nodes of the respective drug may be the mean (or maximum or minimum) distance between the drug targets of the two drugs. The distance between two drug targets may be the length of the minimum length path identified between the two drug targets. The distance between two drugs may be the mean, minimum or maximum of such distances across all pairs of targets comprising a target of drug 1 and a target of drug 2. The mean may conveniently be used as a robust metric.

Any annotation, whether associated with a drug or a drug target, may be in the form of one or more terms of a controlled vocabulary. A controlled vocabulary may be organized as an ontology. In this context, an ontology is a representation of terms that form part of a controlled vocabulary and relationships between them. In the context of the present disclosure, the ontology may be a hierarchical ontology. This may comprise a plurality of hierarchical levels, where each level comprises a plurality of categories, that can each comprise one or more children categories at the next level. Thus, a controlled vocabulary may be represented as a directed acyclic graph, where every node of the graph corresponds to a term of the controlled vocabulary, and edges indicate parent-child relationships between nodes.

Examples of controlled vocabularies organised as hierarchical ontologies include Medical Subject Headings, gene ontology (GO, geneontology.org/), and disease ontology (DO, disease-ontology.org/). Additionally, the Anatomical Therapeutic Chemical (ATC) classification system (see www.who.int/tools/atc-ddd-toolkit/atc-classification) is also organised as a hierarchical classification system and is therefore a hierarchical ontology that can be analysed using any method designed for comparing annotations organised as a hierarchical ontology.

The terms “similarity metric” and “similarity score” (or sometimes just “score) are used interchangeably to refer to values that quantify how similar two values, annotations or sets of values/annotations are to each other. The term “annotations” or “terms” are used interchangeably and refer to words/sets of words that are associated with a drug or drug target. These are typically selected from a controlled vocabulary such as MESH, GO, DO, as described herein.

Metrics of similarity between annotations may include any metrics of similarity known in the art that are applicable to discrete sets in general or to discrete sets organised as ontologies. Metrics of similarity between annotations may be scores between 0 and 1. Similarity metrics that are applicable to discrete sets include the Jaccard index (also referred to as Jaccard similarity), the Dice similarity, the cosine similarity, the Ochiai similarity and the Kulczynski2 metric. The Dice similarity is a score between 0 and 1 calculated as the double of the number of elements shared between two sets, divided by the number of elements in the two sets (number of unique elements across the two sets), see equation (16). The Jaccard similarity is the ratio of the number of features common to both sets to the total number of features in both sets, i.e. (A intersect B)/(A+B−(A intersect B)). The cosine similarity between two vectors is provided as equation (0), where when comparing two sets of annotations each set may be expressed as a vector of length equal to the total number of unique elements across the two sets, with a “1” or “0” for each element depending on whether the element is present or absent in the particular set. The Ochiai similarity measure (also known as Carbo similarity or Cosine coefficient) is calculated as the ratio of the number of features common to both molecules to the square root of the product of the number of features in the two molecules. The Kulczynski2 metric is calculated as the ratio of (the number of features in common to both molecules multiplied by the average size of the total number of features present) divided by the product of the size of the total number of features present (i.e. ((A intersect B)*0.5(A+B))/(A*B)).

Similarity metrics that are designed for ontologies take into account the fact that two terms to be compared may be different but may be related to each other through the ontology. In other words, similarity metrics that are designed for ontologies compare terms by taking into account their relationships in the ontology. Similarity metrics that are designed for ontologies, where the ontologies specify a controlled vocabulary (as is the case for ontologies such as DO, GO and MESH), may also be referred to as “semantic similarity metrics” because they take into account the meaning of the terms to be compared (in the form of their relationships in the ontology). Similarity metrics that are applicable to annotations that form parts of hierarchical ontologies include the Wang, Resnik, Relevance, Jiang and Lin similarity metrics. The Wang similarity metric is described in Wang et al. 2007 and calculated using equations (1), (2), (3), (4a), (4b), (5a) and (5b) provided further below. The Resnick method is described in Resnick 1999. It computes the semantic similarity between two concepts using equation (6). The Lin method is described in Lin 1998. The Lin Similarity Measure is a Node-based Semantic Similarity Measure that is based on information content of the least common subsumer. It is calculated using equations (7), (8) and (9). The Relevance method is described in Schlicker et al. 2006. It combines Resnick and Lin's method and is calculated using equation (10). The Jiang method is described in Jiang and Conrath 1997. It is calculated using equation (11). Each of the above metrics compare pairs of ontology terms. Where multiple ontology terms are associated with one or both of two sets to be compared, multiple pairs of ontology terms can be compared using any of the above metrics, resulting in a plurality of similarities between pairs of annotations of the respective drugs in the combination.

Multiple similarities between pluralities of pairs (each pair comprising an entry associated with one drug and an entry associated with the other drug in the combination—where entries can be annotations, pathways represented as sets of genes, etc.) for a drug combination can be combined using any method known in the art. Methods known in the art for combining a plurality of pairwise similarities obtained by comparing two sets of entries include any method selected from the max, average, rcmax, and best-match average methods. The max method calculates the maximum similarity score over all pairs of terms (see equation 12). The average (avg) method uses the average of similarity scores over all pairs of terms (see equation 13). The rcmax method computes the maximum of (i) the average of maximum similarity on each row, and (ii) the average of maximum similarity on each column (see equation 14)—where rows and columns refer to rows and columns of a matrix where terms annotated to one drug correspond to rows and terms annotated to the other drug correspond to columns, and the values in the matrix are the similarity between the corresponding terms. The best-match average (BMA) method measures the average of maximum similarity scores on each row and column (see equation 15), where rows and columns are as above. In embodiments, any of the above methods, such as the BMA method, is used to combine multiple similarity metrics obtained for respective pairs of targets and/or ontology terms for a pair of drugs.

Multiple predictive features for a drug combination may also be combined by obtaining a summarized value for the plurality of predictive features. Throughout this disclosure, a “summarised value” may be any statistic that summarises a population of values, such as for example the sum, average, median or predetermined percentile (e.g. 1st, 2nd, 5th, 10th, 15th, 85th, 90th, 95th, 98th or 99th percentile) over a plurality of values. A summarised value may comprise a plurality of values, provided that the dimension of the summarised value is lower than the dimension of the values that it summarises. For example, a summarised value may be obtained by dimensionality reduction, such as e.g. using PCA. In embodiments, predictive features are not summarised and each predictive feature (optionally preprocessed such as e.g. by normalisation) is used individually as input to the machine learning model.

Any set of predictive features may be preprocessed prior to being provided as input to a machine learning model. Pre-processing may include one or more of: filtering, log transformation, imputation, standardization, and normalization. Imputation refers to the process of providing values for missing data. Any imputation method known in the art may be used, such as e.g. mean substitution (replacing missing values by the mean of the values for the feature in a dataset, e.g. a training dataset), regression imputation (where a regression model is estimated to predict observed values of a variable based on other variables, and once trained the model can be used to predict missing values of the variable), etc. Filtering may be based may comprise one or more of: removing duplicate entries (e.g. from a training or test dataset), removing entries that have a number or proportion of missing values above a predetermined threshold (e.g. removing any entries where 3 or more of the predictive features are missing prior to any imputation), etc. Standardisation refers to the processing of scaling the value of a feature based on a measure of variability of the feature (typically the standard deviation) in a reference dataset. Normalisation can be performed using any method known in the art including mean centring, obtaining a z score (also known as standard score), min-max scaling (also called unit based normalization, in which a transformer value for each value for a feature is obtained as (x−xmin)/(xmax−xmin), where x is the value of the feature, and xmin and xmax are the minimum and maximum values observed for the feature in a reference dataset). A reference dataset may be a training dataset or a dataset to which the data point to be normalized belongs.

Drug combinations that have been selected using methods as described herein may be validated using one or more assays. An assay may be an in vitro assay, or an in vivo assay. An assay may be an in vitro assay. An in vitro assay may be a cell-based assay or a tissue-based assay. A cell-based assay may be a cytotoxicity assay, a cell proliferation assay, or any assay that measures a cellular phenotype of interest (motility, viability, proliferation, expression, etc.). A tissue-based assay (including tissue culture and organoid-based assays) may be a toxicity assay, or any assay that measures a tissue phenotype of interest (such as e.g. tissue composition, structure, lesions, etc.) Readouts of an in vitro cell or tissue-based assay may be acquired using any bioanalytical instrument known in the art such as e.g. microscopy, flow cytometry, etc. or any omics protocol known in the art. An in vivo assay may characterize any phenotypic or physiological characteristic of interest, such as e.g. tumour growth, viability, physiological function (e.g. function of an organ or system, etc). Readouts of an in vivo assay may comprise physiological measurements, phenotypic measurements (including e.g. behavioral measurements), and omics measurements (e.g. genomics, transcriptomics, proteomics, microbiomics, metabolomics characterization of a subject).

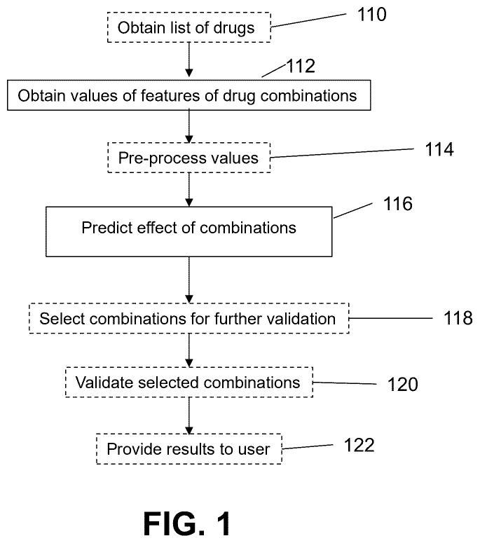

FIG. 1 is a flowchart illustrating a method for predicting the effect of a drug combination according to a general embodiment of the disclosure. At step 110, a list of drugs or drug combinations comprising a first drug and a second drug are obtained. When a list of drugs is obtained, step 110 may further comprise defining pairs of drugs from the list, At step 112, the values of a plurality of features associated with each drug in a combination are obtained. Obtaining values of features associated with a drug combination may comprise, for each of said plurality of features: receiving the value from a database, user interface, or memory, or computing the value based on characteristics of the first and second drugs. Thus, step 110 may further comprise, obtaining, for each drug in the drug combinations to be assessed, a plurality of characteristics from which the values of said plurality of features can be computed. The plurality of characteristics may include one or more of: indication annotations associated with the drugs, one or more drug targets of the drugs, indication annotations associated with one or more targets of the drug, disease annotations associated with one or more targets of the drug, molecular structure of the drugs or information representing the molecular structure of the drugs (e.g. molecular fingerprints), side effect annotations associated with the drugs, mode of action annotations associated with the drugs. Alternatively, when drug combinations are obtained at step 110, the values of the features may also be obtained at step 110, associated with the respective drug combinations. The plurality of features comprises at least one feature that characterises the similarity between one or more indications or diseases associated with one or more targets of the first drug and one or more indications or diseases associated with one or more targets of the second drug. The plurality of features may advantageously comprise at least one feature that characterises the similarity between one or more indications and/or diseases associated with one or more targets of the first drug and one or more indications and/or diseases associated with one or more targets of the second drug. Indications may be provided as Medical Subject Headings terms associated with the one or more targets of the drugs. Diseases may be provided as disease ontology terms associated with the one or more targets of the drugs. The plurality of features can comprise: one or more drug similarity features, one or more target similarity features, and one or more indication similarity features, as described herein. For example, the plurality of features can comprise: at least one feature that characterises the similarity between one or more indications associated with one or more targets of the first drug and one or more indications associated with one or more targets of the second drug, wherein indications are provided as Medical Subject Headings terms associated with the one or more targets of the drugs; a side effect similarity, a mode of action similarity, a molecular structure similarity, a pathway similarity, a disease association similarity that characterises the similarity between one or more diseases associated with one or more targets of the first drug and one or more indications or diseases associated with one or more targets of the second drug, and a metric of closeness of the targets of the first and second drugs on a protein-protein interaction network, and an indication similarity metric based on the drugs that characterises the similarity between indication annotations associated with the first and second drugs, wherein the indication annotations are provided as anatomical therapeutic chemical (ATC) codes, and wherein the indication similarity metric based on the drugs comprises a similarity metric that quantifies the similarity between multiple levels of the ATC codes for the first and second drugs. The indication similarity metric based on the drugs may further comprise a similarity metric that compares the first level of the ATC codes for the first and second drugs, wherein the similarity metric that compares the first level of the ATC codes for the first and second drugs is a set of binary features that indicate whether the first and second drugs are in each of a plurality of pairs of first level ATC categories, optionally wherein the plurality of pairs of first level ATC categories include LL, NN, and optionally one or more of JJ, CC, JS, DR, DS, CJ, AA, AM, AN, and BN.

At step 114, the values obtained at step 11 may be pre-processed. This may comprise one or more of: missing values imputation, filtering, standardisation and normalisation. Missing value imputation may use any algorithm known in the art, such as e.g. KNN. The missing value imputation process may use information from a training and/or test dataset that has been used to train the model. Filtering may exclude combinations that have a proportion of missing features above a predetermined threshold (e.g. more than 2 missing features amongst 8 or 9 predictive features). Standardisation and normalisation may use information from a training and/or test dataset that has been used to train the model. For example, the mean value and standard deviation of a feature across a training and/or test dataset may be used to normalise and standardise the values for the feature in a combination for which an effect is to be predicted.

At step 116, the values obtained at step 112 or step 114 are used to predict the effect of at least one drug combination obtained at step 110. This uses a machine learning model that has been trained to predict whether a drug combination is likely synergistic, using training data comprising for each of a plurality of training drug combinations: (i) the values of said plurality of features and (ii) an indication of whether the drug combination is synergistic. The machine learning model may have been trained to classify drug combinations between at a plurality of categories comprising a synergistic category and a non-synergistic category, and step 116 may comprise determining whether the drug combination is likely to be in the synergistic category.

At step 118, one or more of a plurality of combinations for which an effect was predicted at step 116 is/are selected for further validation. For example, the top x predicted combinations (top x combinations with the highest probability of synergy) may be selected. Alternatively, one or more combinations may be selected from the top x combinations. For example, factors such as prior knowledge and availability may be used to select drug combinations from the top predicted combinations. The value of x may depend on practical factors such as testing ability. Similarly, the predicted combinations with probability of synergy above a threshold y, or a subset of these may be selected. For example, factors such as prior knowledge and availability may be used to select drug combinations from the top predicted combinations with probability above threshold y. Again the value of threshold y may depend on practical factors and the distribution of probabilities predicted for a set of drugs being screened.

At step 120, the selected combinations are validated using one or more in vitro and/or in vivo assays (e.g. preclinical and clinical trials). The results of any of the preceding steps may be provided to a user at step 122.

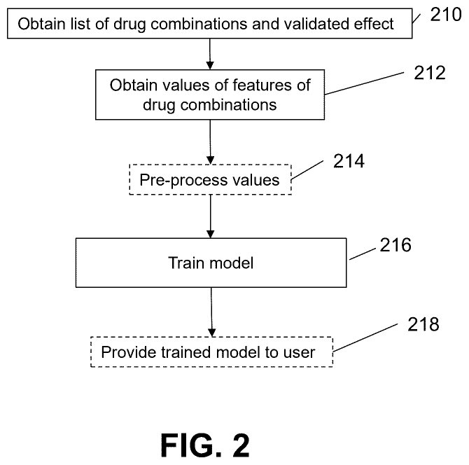

FIG. 2 is a flowchart illustrating a method for providing a tool for predicting the effect of a drug combination according to an embodiment of the disclosure. Step 210 comprises obtaining, for each of a plurality of training drug combinations comprising a first drug and a second drug: (i) the values of a plurality of features associated with the drug combination or characteristics of the drugs from which such values can be obtained; and (ii) information indicative of whether the drug combination is synergistic. At step 212, the values of a plurality of features associated with the drug combinations are computed when these were not already obtained at step 210. The plurality of features comprises at least one feature that characterises the similarity between one or more indications or diseases associated with one or more targets of the first drug and one or more indications or diseases associated with one or more targets of the second drug.

At step 214, the training data obtained at steps 210 and 212 is optionally preprocessed. This may comprise one or more of: filtering, missing value imputation, normalisation and standardisation. For example, the data may be filtered to exclude any drug combination that has more than a predetermined number or proportion of missing values. For example, drug combinations with more than two features missing may be removed. The optionally filtered data may then be subject to data imputation to obtain a full dataset (i.e. no missing values). For example, this may be performed using a KNN algorithm. The data may then be normalised (subtracting the mean across observations on feature by feature basis) and/or standardised (dividing by the standard deviation across observations on feature by feature basis).

At step 216, the values obtained at steps 212 or 214 are used to train a machine learning model to predict whether a drug combination is likely synergistic, using the training data obtained at steps 210 and 212. The machine learning model is trained to take as input the values of said plurality of features for a drug combination and to produce as output an indication of whether the rug combination is likely to be synergistic. This may comprise performing feature selection, for example by determining the performance of each of a plurality of models trained with respective subsets of the plurality of features, and selecting the smallest model with performance within a predetermined tolerance of the performance of the best model. Performance may be assessed using any metric known in the art, such as F1 score, accuracy, balanced accuracy, precision, recall, etc. In the present setting metrics such as F1 score and balanced accuracy are believed to be advantageous because drug combination training data is likely to be unbalanced (as many more non-synergistic combinations than synergistic combinations are known).

At step 218 the trained model is provided to a user, data store or computing device. This can be used in a method as described by reference to FIG. 1.

The methods described herein are computer-implemented unless context specifies otherwise (such as e.g. where measurement steps and/or wet steps are involved). Thus, the methods described herein are typically performed using a computer system or computer device. Any reference to an action such as “obtaining”, “processing”, “determining” may therefore refer to a processor performing the action, or a processor executing instructions that cause the processor to perform the action. Indeed, the methods of the present invention comprising at least the processing of drug annotations as large ontologies or drug structural similarities, especially on the scale of tens or hundreds of thousands of drug combinations, is such that it cannot be performed in the human mind. As used herein, the terms “computer system” of “computer device” includes the hardware, software and data storage devices for embodying a system or carrying out a computer implemented method. For example, a computer system may comprise one or more processing units such as a central processing unit (CPU) and/or a graphical processing unit (GPU), input means, output means and data storage, which may be embodied as one or more connected computing devices. Preferably the computer system has a display or comprises a computing device that has a display to provide a visual output display (for example in the design of the business process). The data storage may comprise RAM, disk drives or other computer readable media. The computer system may include a plurality of computing devices connected by a network and able to communicate with each other over that network. For example, a computer system may be implemented as a cloud computer. The term “computer readable media” includes, without limitation, any non-transitory medium or media which can be read and accessed directly by a computer or computer system. The media can include, but are not limited to, magnetic storage media such as floppy discs, hard disc storage media and magnetic tape; optical storage media such as optical discs or CD-ROMs; electrical storage media such as memory, including RAM, ROM and flash memory; and hybrids and combinations of the above such as magnetic/optical storage media.

The methods described herein find application in a variety of contexts. For example, the methods described herein can be used to determine which of a plurality of candidate drug combinations (e.g. a plurality of drug combinations comprising a known disease modifying drug for a particular disease and an additional drug that has not been tested clinically in combination with the known disease modifying drug and/or has not been tested clinically for the treatment of the particular disease) are likely to produce a synergistic effect, and/or to select one or more of a plurality of candidate drug combinations for inclusion in a set of drug combinations to be further characterised (e.g. experimentally validated) for the treatment of a particular disease. For example, candidate combinations that are predicted as likely to produce a synergistic effect may be selected. For example, a predetermined number of drug combinations may be selected as the drug combinations with the highest predicted probability of synergy. As another example, a predetermined number of drug combinations may be selected as drug combinations amongst the top x (e.g. top 10, top 20) drug combinations with the highest predicted probability of synergy, for example based on additional literature support, expert knowledge, availability for testing, etc. As another example, a predetermined number of drug combinations may be selected as drug combinations amongst set of drug combinations with predicted probability of synergy above a predetermined threshold (e.g. 0.20 or more, 0.25 or more, 0.30 or more, etc), for example based on additional literature support, expert knowledge, availability for testing, etc. As another example, all drug combinations with predicted probability of synergy above a predetermined threshold (e.g. 0.20 or more, 0.25 or more, 0.30 or more, etc) may be selected for further characterisation.

Because the methods are able to prioritise promising drug combinations, they can significantly reduce the costs and time associated with screening of drug combinations. This is extremely valuable in practice as drug combinations are cumbersome and there is a combinatorial explosion of the number of combinations that can be tested. However, if effective combinations can be identified this can be extremely valuable since an improved therapy can be offered to patients without the need to develop and characterise completely new compounds.

Thus, also described herein is a method of screening a plurality of candidate drug combinations, the method comprising: obtaining a plurality of candidate drug combinations; predicting the effect of each of the plurality of candidate drug combinations using a method as described herein; and selecting a subset of the plurality of candidate drug combinations for experimental testing using the results of the predicting. Similarly, also described herein is a method of providing a candidate drug combination for the treatment of a particular disease, the method comprising: obtaining a plurality of candidate drug combinations each comprising at least one drug that is a known disease modifying drug for the particular disease, and identifying at least one drug combination of the plurality of candidate drug combinations as likely to have a synergistic effect in the treatment of the particular disease using a method as described herein.

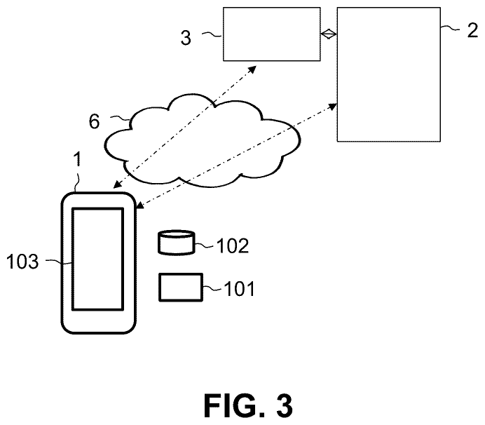

FIG. 3 shows an embodiment of a system for predicting the effect of a drug combination, for providing a tool for predicting the effect of a drug combination, and/or for providing a drug combination according to the present disclosure. The system comprises a computing device 1, which comprises a processor 101 and computer readable memory 102. In the embodiment shown, the computing device 1 also comprises a user interface 103, which is illustrated as a screen but may include any other means of conveying information to a user such as e.g. through audible or visual signals. The computing device 1 is communicably connected, such as e.g. through a network, to one or more databases 2 storing drug data (values of a plurality of predictive features associated with drugs, and optionally drug combination effect ground truth label data). The one or more databases 2 may further store one or more of: one or more machine learning algorithms, training data, parameters (such as e.g. parameters of machine learning model, feature selection algorithm, data preprocessing methods, etc.), etc. The computing device may be a smartphone, tablet, personal computer or other computing device. The computing device is configured to implement a method as described herein. In alternative embodiments, the computing device 1 is configured to communicate with a remote computing device (not shown), which is itself configured to implement a method of as described herein. In such cases, the remote computing device may also be configured to send the result of the method to the computing device. Further, the various steps of the methods described herein may be split between the computing device 1 and the remote computing device. Communication between the computing device 1 and the remote computing device may be through a wired or wireless connection, and may occur over a local or public network 6 such as e.g. over the public internet. The computing device 1 may also be connected to drug combination testing means 3 (such as e.g. assay systems for in vitro testing of a drug combination). The drug combination testing means may be in wired connection with the computing device 1, or may be able to communicate through a wireless connection, such as e.g. through WiFi and/or over the public internet, as illustrated. The connection between the computing device 1 and the drug combination testing means 3 may be direct or indirect (such as e.g. through a remote computer). The drug combination testing means 3 may include any assay system configured to acquire biological data indicative of the effect of a candidate drug combination.

The following is presented by way of example and is not to be construed as a limitation to the scope of the claims.

### Examples

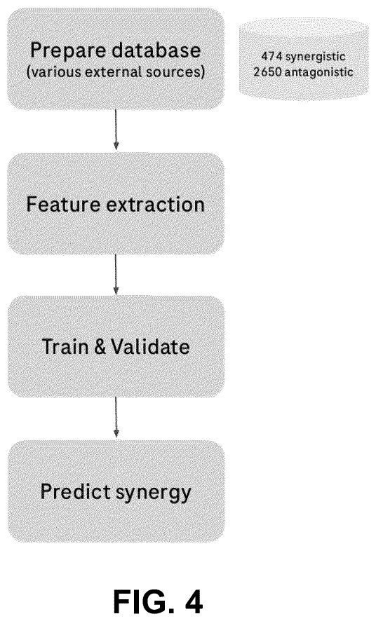

Exemplary methods of the disclosure will now be described. In particular, the examples below demonstrate the use of the methods of the invention to find a ‘high potential drug combination’ for improving the treatment of MS. The general workflow followed in these examples is illustrated on FIG. 4.

### Materials and Methods

Data. Publicly available databases (FDA orange book and DrugBank) provided data on 474 synergistic and 2,650 antagonistic drug combinations (FIG. 4, step 1). The FDA orange book and DrugBank contained data from 3133 drug combinations, of which 474 were found to have a synergistic effect and 2659 were found to have an antagonistic effect through clinical and/or preclinical/research studies. The data was downloaded from DrugComb database (Liu et al. 2020), where it was annotated as synergistic (combinations from the FDA orange book) or non-synergistic (combinations from DrugBank). Synergistic drugs were ones where effect 1+2>effect 1+effect 2. Additive drugs (effect 1+2=effect1+effect2) were not considered here. Antagonist drugs were drugs where effect 1+2<effect 1+effect 2 or sideeffect 1+2>sideeffect1+sideeffect2. Drugs in a tested pair were considered synergistic if the effect of the combination was better than the sum of the effect of the two drugs in isolation. An example of this is the combination of chlorpromazine and pentamidine. Neither of these drugs showed any anti-tumour activity when tested alone, but the two drugs together prevented tumour growth even more effectively than paclitaxel (a known cytotoxic drug). Drugs were considered antagonistic if their effect in combination was not better than the sum of their effects in isolation and/or their side effects in combination were worse than the sum of their side effects in isolation (i.e. effect 1+2<effect 1+effect 2 or sideeffect 1+2>sideeffect1+sideeffect2). An example of this is sorafenib and pegylated interferon α2-b, which together produce more cutaneous side effects than using individual agents. Of these, 5 drug combinations contained one existing MS disease modifying therapy as one of the members of the pair. All 5 pairs were included in unseen data used for model testing, together with a randomly selected set of pairs representing 20% of the data. For each drug pair, a feature vector was built containing 113 features (FIG. 4, Step 2):
Synergy combination: this is a binary value 0 (no synergy) or 1 (synergy) (ground truth label for model training);
Pathway similarity: score=Dice similarity computed using the R package BioCor (Revilla Sancho L, 2023);
MESH similarity: score=semantic similarity computed using the R package meshes (Yu G, 2018);
ATC similarity: score=Jaccard similarity;
MOA similarity: score=Jaccard similarity;
Disease ontology similarity: score=semantic similarity computed using R package DOSE (Yu G, 2015);
Network separation: score=min distance between clusters, computed using R package igraph (Csárdi G et al. 2023);
Side effect similarity: overlapping side effects (intersection/union);
Molecular structure similarity: score=Tanimoto similarity computed using R package fingerprint (Guha R, 2018);
First level ATC: 105 features describing all combinations of the first level ATC codes, one hot encoded as binary values.
   Each of these is described in more detail below.

Predictive features-drug similarity features. Three different drug similarity features were used: molecular structure similarity, mode of action similarity, and side effects similarity.

Side effect similarity is calculated as the number of side effects in common (intersection) divided by the number of unique side effects listed for either of the two drugs (union) (Jaccard similarity). Side effects are extracted from OnSIDES (ON label SIDE effects resource, available at tatonettilab.org/onsides-side-effects-extracted-from-fda-structured-product-labels/—this database lists adverse reactions and boxed warnings extracted from the FDA structured product labels from DailyMed; the data is downloaded as tabular data) and compared by exact matching (DailyMed uses a unified vocabulary). For example, in a situation where drug 1 has the side effects “drowsiness, diarrhea, vomiting” and drug 2 has the side effects “Drowsiness, Thirst”, the intersection is =1 (vomiting), and the union is =4 (drowsiness, diarrhea, vomiting, thirst), so the side effect similarity is ¼ (i.e. 0.25). Side effect similarity is a score between 0 and 1.

Mode of action similarity is calculated as the number of modes of action terms in common (intersection) divided by the number of unique mode of action terms listed for either of the two drugs (union). Mode of action terms are extracted from DGIdb (the Drug Gene Interaction database, Freshour et al. 2020) and compared by exact matching (the mode of action is specified in DGIdb using a predefined vocabulary, see www.dgidb.org/interaction_types; each drug can be associated with one or more mode of action terms, for example each associated with respective targets). For example, in a situation where drug 1 has the mode of action terms “inhibitor, blocker” and drug 2 has the mode of action terms “antibody, inhibitor”, the intersection is =1 (inhibitor), and the union is =3 (inhibitor, blocker, antibody), so the side effect similarity is ⅓ (i.e. 0.33). Mode of action similarity is a score between 0 and 1.

Molecular structure similarity was calculated as the Tanimoto similarity as implemented in the R package fingerprint. The Tanimoto similarity is is a score between 0 and 1 that quantifies how similar two small molecules are to each other. Any molecular structure similarity metric of this type known in the art may be used instead. For example, the fingerprint package also includes implementations of the Dice, modified Tanimoto, Simple, Jaccard, Cosine and Achai similarity, amongst others. The similarity between two molecules can be calculated based on their respective fingerprints. A fingerprint can be specified as a binary vector (vector of 0s and 1s). A fingerprint may characterize the 2D structure of a molecule (2D-fingerprint). There are multiple methods to determine binary molecular fingerprints. For example, the extended-connectivity fingerprint ECFP4 is commonly used and is described in Rogers and Hahn (2010). Any method known in the art to calculate a molecular fingerprint may be used. In particular, any method known in the art to calculate a binary 2D-fingerprint may be used. This looks at the presence of specific circular substructures around each atom in a molecule. In the present example, the Rcpi R package (Cao et al. 2015) was used to obtain fingerprints. The getSmiFromChEMBL function in this package takes as input a CHEMBL identifier and produces as output a SMILES strings. The simplified molecular-input line-entry system (SMILES) is a specification in the form of a line notation for describing the structure of chemical species. This can then be turned into FP4 fingerprints using function extractDrugOBFP4. Any similarity metrics known in the art for the purpose of comparing molecular fingerprints may be used, including any of the molecular similarity measures reviewed in Maggiora and Shanmugasundaram, 2011, such as e.g.: Tanimoto, Dice, Modified Tanimoto, Cosine similarity, Ochiai, Kulczynski2. The Tanimoto similarity (also referred to as Tanimoto coefficient, or Jaccard similarity) is the most commonly used measure of similarity or chemical distance between two compounds is the ratio of the number of features common to both molecules to the total number of features, i.e. (A intersect B)/(A+B−(A intersect B)). The Dice coefficient is the number of features in common to both molecules relative to the average size of the total number of features present, i.e. (A intersect B)/0.5 (A+B). The modified Tanimoto method is a modification of the Tanimoto method that aims to be more accurate for larger molecules. The cosine similarity between any two vectors A, B of size n is calculated as:

$$S ( A , B ) = \frac{A \cdot B}{\vert \vert A \vert \vert ‖ B ‖} = \frac{{\sum }_{i = 1}^{n} {A}_{i} {B}_{i}}{\sqrt{{\sum }_{i = 1}^{n} {A}_{i}^{2} {\sum }_{i = 1}^{n} {B}_{i}^{2}}} \qquad ( 0 )$$

The Ochiai similarity measure (also known as Carbo similarity or Cosine coefficient) is calculated as the ratio of the number of features common to both molecules to the square root of the product of the number of features in the two molecules. The Kulczynski2 metric is calculated as the ratio of (the number of features in common to both molecules multiplied by the average size of the total number of features present) divided by the product of the size of the total number of features present (i.e. ((A intersect B)*0.5 (A+B))/(A*B)).

Large molecules (such as antibodies) were assigned a missing value (NA) for this predictive feature. However, similarity metrics for large molecules such as antibodies can be computed using metrics based on one or more of primary sequence, secondary structure (e.g. Musnier et al. 2022) and 3D structure (e.g. RMSD of all or part of the structure).

Predictive features—target similarity features. Five different target similarity features were used: pathway similarity, target protein similarity, disease association similarity, co-expression, and closeness on protein-protein interaction network (PPI).

Target protein similarity is a score between 0 and 1 based on the known targets of the drugs on the combination, calculated as a Jaccard similarity, i.e. target_protein_similarity=intersection (drug1_targets, drug2_targets)/union (drug1_targets, drug2_targets). This feature was not used in the final model.

Co-expression is a score between 0 and 1 based on the average expression of all respective targets of the drugs in all tissues. The average expression of each gene in all tissue were downloaded from www.proteinatlas.org. The score was calculated as correlation (drug1_targets_expression, drug2_targets_expression), where correlation is the Pearson correlation coefficient and drug1_targets_expression is a vector of average expression of the targets of drug1, comprising one value per tissue (and similarly for drug2_targets_expression). This feature was not used in the final model.

Disease association similarity is a score between 0 and 1 based on Disease Ontology of the target(s) of the drugs. For example, a drug 1 with target CD40 may be associated with the ontology term “CD40 associated diseases”, which may be associated with “cancer” and “MS”. A drug 2 with target CD20 may be associated with the ontology term “CD20 associated diseases”, which may be associated with “viral infection” and “MS”. The similarity between drugs 1 and 2 will reflect the semantic similarity between these two ontology terms. The disease association similarity was calculated using the R package DOSE (Yu G, 2015). DOSE is an R/Bioconductor package for disease ontology semantic and enrichment analysis. Disease Ontology is a consistent scheme for annotation of human genes in a disease perspective. The disease ontology is available in the DO database (disease-ontology.org; Schriml et al., 2012), and gene DO annotations are available at the Disease and Gene Annotations database (DGA, dga.nubic.northwestern.edu; Peng et al. 2013). As an ontology, the DO is organized as a directed acyclic graph, and as such annotations can be compared using semantic similarity algorithms. The DOSE packages provides an algorithm for computation of the semantic similarity between genes based on their annotated DO terms (geneSim function). The geneSim function can compute a similarity between pairs of genes provided as entrez gene identifiers, using one of 5 similarity metrics (Wang, Resnik, Relevance, Jiang and Lin), and using one of 4 methods for aggregating semantic similarity scores for multiple DO terms associated with a gene (max, avg, rcmax, BMA). The Wang method was used in the present examples to obtain pairwise similarities between DO terms but any of the above methods could be used instead. The BMA method was used in the present examples to aggregate multiple pairwise similarities, but any of the above aggregation methods may also be used. Any of these aggregation methods may also be used to combine similarities for multiple pairs of targets (i.e. a similarity is calculated for each pair comprising a target of drug 1 and a target of drug 2, and the resulting similarities are aggregated using any of the above methods). In the present examples the BMA method was used for this but any of the above aggregation methods may be used instead. The Wang method is described in Wang et al. 2007. It calculates the similarity between genes G1 and G2 as:

$$S i m ( G 1 , G 2 ) = \frac{{\sum }_{1 \leq i \leq m} S i m ( g {o}_{1 i} , {GO}_{2} ) + {\sum }_{1 \leq j \leq n} S i m ( g {o}_{2 j} , {GO}_{1} )}{m + n} \qquad ( 1 )$$

where GO₁={go₁₁, go₁₂, . . . , go₁m} and GO₂={go₂₁, go₂₂, . . . , go₂ₙ} are the ontology (e.g. GO, DO, etc, as the case may be) term sets annotated to genes G1 and G2, respectively, Sim(go,GO) is the semantic similarity between one term go and a set of terms GO, defined as:

$$S i m ( go , GO ) = {max}_{1 \leq i \leq k} ( {S}_{G O} ( go , {go}_{i} ) ) \qquad ( 2 )$$

where goi is a term in the set GO and k is the size of the set GO, and the similarity between two terms A, B (e.g. A is go and B is goi in the equation above) defined by respective directed acyclic graphs DAG_{A}=(A, T_{A}, E_{A}) and DAG_{B}=(B, T_{B}, E_{B}) is defined as:

$${S}_{G O} ( A , B ) = \frac{{\sum }_{t ∈ {T}_{A} ∩ {T}_{B}} ( {S}_{A} ( t ) + {S}_{B} ( t ) )}{S V ( A ) + S V ( B )} \qquad ( 3 )$$

where terms t are terms related to terms A and B, respectively, T_{A} is the set of GO terms in DAG_{A}, including term A and all of its ancestor terms in the graph, and E_{A} is the set of edges (semantic relations) connecting the GO terms in DAG_{A} (and similarly for corresponding terms in DAG_{B}), SV(A) is the semantic value of GO term A, and SV(B) is the semantic value of GO term B, provided by:

$$S V ( A ) = {\sum }_{t ∈ {T}_{A}} {S}_{A} ( t ) \qquad ( 4 a )$$

$$SV ( B ) = {\sum }_{t ∈ {T}_{B}} {S}_{B} ( t ) \qquad ( 4 b )$$

where the S-value of each term in each DAG is defined as:

$$\begin{matrix}\{ {S}_{A} ( A ) = 1 {S}_{A} ( t ) = \{ {t}^{′} ∈ childrenof ( t ) \} & if t \neq A\end{matrix} \qquad ( 5 a )$$

$$\begin{matrix}\{ {S}_{B} ( B ) = 1 {S}_{B} ( t ) = \{ {t}^{′} ∈ childrenof ( t ) \} & if t \neq B\end{matrix} \qquad ( 5 a )$$

where wₑ is the semantic contribution factor for edge e∈E_{A} (respectively E_{B}) linking term t with its child term t′, with 0<wₑ<1. The semantic value of a GO term is the aggregate semantic contribution of all terms in a DAG representing this GO term. The weights wₑ (semantic contribution factors) are set to fixed values depending on the type of relations. For example, “is-a” may be associated with a weight of 0.8, and “part-of” may be associated with a weight of 0.6.

The Resnick method is described in Resnick 1999. It computes the semantic similarity between two concepts as:

$$s i m ( {c}_{1} , {c}_{2} ) = {max}_{c ∈ S ( {c}_{1} , {c}_{2} )} [ - log p ( c ) ] \qquad ( 6 )$$

where S(c1,c2) is the set of concepts that subsume both c1 and c2 and p is a function p:C→[0,1] such that for any c∈C, p(c) is the probability of encountering an instance of concept c, and the top node probability is 1. This can be computed as a relative frequency p(c)=freq(c)/N, where N is the total number of nouns observed and freq(c)=Σ_{n∈words(c)} count(n) where words(c) is the set of words subsumed by concept c.

The Lin method is described in Lin 1998. The Lin Similarity Measure is a Node-based Semantic Similarity Measure that is based on information content of the least common subsumer. It is defined as:

$$s i {m}_{Lin} ( {c}_{1} , {c}_{2} ) = \frac{2 * s i m ( {c}_{1} , {c}_{2} )}{IC ( {c}_{1} ) + IC ( {c}_{2} )} \qquad ( 7 )$$

where sim (c1,c2) is the Resnick similarity as defined in equation (6) and IC(c₁) and IC(c₂) are the information content of the concepts c1 and c₂, defined as:

$$IC ( c ) = - log p ( c ) \qquad ( 8 )$$

where p(c) is defined as explained above as:

$$p ( c ) = {n}_{{c}^{′}} / N \vert {c}^{′} ∈ \{ c , children of c \} \qquad ( 9 )$$

where n_{c′} is the number of term c′ and N is the total number of terms in the GO corpus. Note that the Resnick similarity is equal to the information content for the most informative common ancestor (MICA).

The Relevance method is described in Schlicker et al. 2006. It combines Resnick and Lin's method and is defined as:

$$s i {m}_{Rel} ( {c}_{1} , {c}_{2} ) = \frac{2 * sim ( {c}_{1} , {c}_{2} ) ( 1 - p ( MICA ) )}{IC ( {c}_{1} ) + IC ( {c}_{2} )} \qquad ( 10 )$$

where sim (c1,c2) is the Resnick similarity as defined in equation (6) and IC(c₁) and IC(c₂) are the information content of the concepts c₁ and c₂ as defined in equations (8)-(9) and MICA is the most informative common ancestor, which is the closest common ancestor term between the two terms that are compared.

The Jiang method is described in Jiang and Conrath 1997. It is defined as:

$$s i {m}_{Jiang} ( {c}_{1} , {c}_{2} ) = 1 - min ( 1 , IC ( {c}_{1} ) + IC ( {c}_{2} ) - s * IC ( MICA ) ) \qquad ( 11 )$$

where MICA is the most informative common ancestor as above.

The max method calculates the maximum similarity score over all pairs of terms (equation 12), the avg method uses the average of similarity scores over all pairs of terms (equation 13), the remax method computes the maximum of (i) the average of maximum similarity on each row, and (ii) the average of maximum similarity on each column (equation 14), and the best-match average (BMA) method measures the average of maximum similarity scores on each row and column (equation 15). Thus, any of the following equations can be used to combine semantic similarity scores over pairs of terms between two term sets (where the pairwise similarity scores are calculated using any of the metrics above):

$$s i {m}_{ma x} ( {g}_{1} , {g}_{2} ) = {max}_{1 \leq i \leq m , 1 \leq j \leq n} s i m ( g {o}_{1 i} , {go}_{2 j} ) \qquad ( 12 )$$

$$si {m}_{avg} ( {g}_{1} , {g}_{2} ) = \frac{{\sum }_{j = 1}^{n} {\sum }_{i = 1}^{m} S i m ( g {o}_{1 i} , {go}_{2 j} )}{m * n} \qquad ( 13 )$$

$$si {m}_{rcm ax} ( {g}_{1} , {g}_{2} ) = max ( \frac{{\sum }_{i = 1}^{m} {max}_{1 \leq j \leq n} sim ( {go}_{1 i} , {go}_{2 j} )}{m} , \frac{{\sum }_{j = 1}^{n} {max}_{1 \leq i \leq m} s i m ( g {o}_{1 i} , {go}_{2 j} )}{n} ) \qquad ( 14 )$$

$$si {m}_{BMA} ( {g}_{1} , {g}_{2} ) = \frac{{\sum }_{i = 1}^{m} {max}_{1 \leq j \leq n} sim ( {go}_{1 i} , {go}_{2 j} ) + {\sum }_{j = 1}^{n} {max}_{1 \leq i \leq m} sim ( {go}_{1 i} , {go}_{2 j} )}{m + n} ) \qquad ( 15 )$$

Pathway similarity is a score between 0 and 1 computed as a Dice similarity based on KEGG pathways (www.genome.jp/kegg/pathway.html). Note that any other pathway database annotation may be used for this. In particular, both Gene ontology (GO) and Reactome annotations were tried. For example, consider drug 1 associated with pathways “immune system” and “cell proliferation” and drug2 associated with pathways “interferon-1 activation” and “cell apoptosis”, the score will reflect the similarity between the “cell proliferation” and “apoptosis” pathways, as well as the “interferon-1 activation” and “immune system” pathways. Pathway associations were based on the drug targets, i.e. a drug was deemed to be associated with any pathway that any of its targets is annotated to. Kegg or Reactome pathway similarity scores were calculated using the R package BioCor (Revilla Sancho 2023). This computes the similarity between two genes specified by their entrez gene identifiers, by computing the Dice similarities between each pair of pathways that the genes are involved in, and combining the multiple similarity values. Combining the multiple similarities between pairs of pathways in two sets of pathways can be done using one of the max method which calculates the maximum similarity score over all pairs of pathways (see equation 12, but where “go” now refers to pathways and sim are Dice similarities between pairs of pathways), the avg method which uses the average of similarity scores over all pairs of pathways (see equation 13, but where “go” now refers to pathways and sim are Dice similarities between pairs of pathways), the rcmax method which computes the maximum of (i) the average of maximum similarity on each row, and (ii) the average of maximum similarity on each column (see equation 14, but where “go” now refers to pathways and sim are Dice similarities between pairs of pathways), the best-match average (BMA) method which measures the average of maximum similarity scores on each row and column (see equation 15, but where “go” now refers to pathways and sim are Dice similarities between pairs of pathways), the reciprocal method (described in Tao et al. 2007, Equation (3)) which computes the double of the sum of the reciprocal maximal similarities above a threshold divided by the number of elements in the two pathway sets being compared

$$( i . e . sim ( A , B ) = \frac{2 * {\sum }_{( {a}_{i} , {b}_{i} ∈ P , si m ( {a}_{i} , {b}_{i} ) \geq y )} s i m ( {a}_{i} , {b}_{i} )}{( \vert A \vert + \vert B \vert )}$$

where t is a threshold set to remove pairs with low similarities, A and B are two sets of pathways with pathways ai, bi, containing |A| and |B| numbers of pathways, P are all reciprocal pairs of pathways (ai,bi), and sim are Dice similarities between pairs of pathways), or the rcmax.avg method which computes the sum of the max values by rows and columns divided by the number of columns and rows. The BMA method was used in the present examples but any other method could be used. The Dice similarity is a score between 0 and 1 calculated as the double of the number of elements shared between two groups (i.e. number of genes that are common between two pathways), divided by the number of elements in the two groups (number of unique genes in total across the two pathways), i.e.:

$$D = \frac{2 \vert X ∩ Y \vert}{\vert X \vert + \vert Y \vert} \qquad ( 16 )$$

where X and Y are two sets to be compared (in this case, two pathways defined by a set of genes involved in the pathway). Note that the Dice and Jaccard similarity metrics are related to each other and it is possible to convert any Dice similarity value to a Jaccard similarity value. Indeed, the corresponding Jaccard index is J=D/(2−D). Conversely, D=2J/(1+J). For GO annotations, only pathway annotations were used and similarities were calculated using the GOSemSim software (Yu et al. 2010). This uses one of 5 similarity metrics (Wang, Resnik, Relevance, Jiang and Lin), and one of 4 methods for aggregating semantic similarity scores for multiple GO terms associated with a gene or pairs of genes (max, avg, rcmax, BMA). In the present examples the Wang and BMA methods were used.

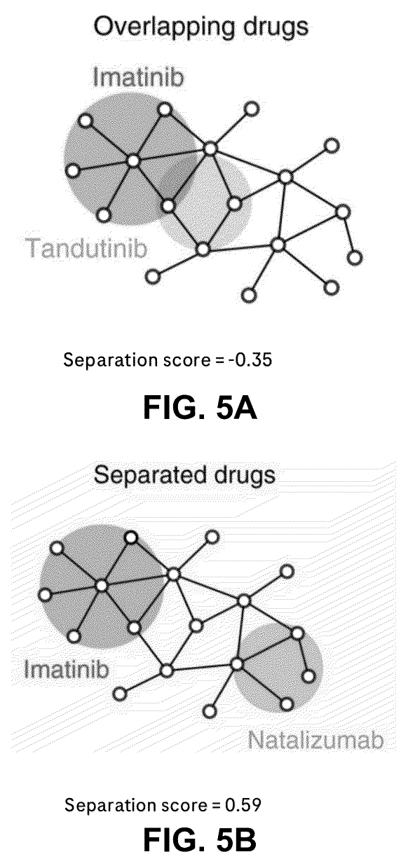

The closeness on protein-protein interaction network (PPI) was calculated as a separation score that quantifies the distance between clusters comprising proteins targeted by the drug, in a PPI. The STRING network (string-db.org) was used as the PPI in these examples. STRING (Szklarczyk et al. 2018) is a database that collects protein-protein functional interaction information from multiple data sources, Including interactions between direct pathway members and within protein complexes. This is illustrated on FIGS. 5A-5B which illustrate two drugs with overlapping target clusters (FIG. 5A) and two drugs with separated target clusters (FIG. 5B), with corresponding closeness scores. Drug targets were obtained from DGIdb (www.dgidb.org) and located on the STRING network. A “cluster” is simply defined as a set of drug targets on the network. The closeness score was calculated as the mean distance between the drug targets of the two drugs using the igraph package to calculate graph-based distances. The distances function in iGraph calculates the length of all the shortest paths from or to selected vertices in a network. In other words, all pairwise distances between a target of drug 1 and a target of drug 2 were calculated (length of minimum length path identified) and the mean of these distances was used as the closeness metric. Note that the minimum or maximum distances could be used instead, as the mean, min and max distances were found to be correlated. Alternatively, any score that can be used to quantify a distance between subnetworks (clusters) in a network may be used.

Predictive features—Indication similarity features. Three different indication similarity features were used: medical heading subjects (MESH) similarity, and ATC similarity, where ATC similarity was calculated in two ways: Jaccard similarity, and first level similarity.

Medical subject headings (MESH) similarity is a score between 0 and 1 quantifying the semantic similarity between indications associated with the target(s) of a drug. This was calculated using the R package meshes. MeSH is a controlled vocabulary used by the National Library of Medicine to manually index articles for MEDLINE/PubMed (see www.nlm.nih.gov/mesh/meshhome.html). The meshes package associates MeSH terms with entrez gene identifiers and provides semantic similarity metrics between genes and groups of genes. These were used to obtain similarity metrics between drugs based on the entrez gene identifiers of their targets. Any of the metrics described above to compare ontologies may be used in this context, including e.g. the Resnick method, the Lin method, the Relevance method, the Jiang method and the Wang method. All of these are implemented in the meshes package. The Wang method was used in the present examples but any of the above methods may be used. Further, any of the above aggregation methods may be used to combine similarities for multiple pairs comprising a target of drug 1 and a target of drug 2. The BMA method was used in the present examples. The meshes package offers 3 methods to associate genes with MeSH terms: gendoo, gene2pubmed, RBBH. The gendoo method is a text-mining based method described in Nakazato et al. 2009. The present inventors selected this method in these examples because it is believed to be more comprehensive and likely to return results for more drugs than other methods. The gene2pubmed method uses the results of a manual curation by NCBI teams. The Reciprocal Blast Best Hit (RBBH) method is based on sequence homology with BLASTP search (E-value<10-50).

Anatomical Therapeutic Chemical (ATC) codes are unique codes associated with a drug according to a classification system that indicates the organ or system that the drug works on and how the drug works. The ATC system is maintained by the World Health Organization (WHO). According to this classification system, each active substance is classified in a hierarchy with 5 different levels. At the first level the classification distinguishes between 14 main anatomical/pharmacological groups defined in Table 1. The second level indicates the therapeutic or pharmacological subgroup, and is provided as 2 digits. The third and fourth levels indicate chemical, pharmacological or therapeutic subgroups. They are each specified as one letter. The fifth level uniquely identifies the chemical substance amongst the group.

For example, the active substance ocrelizumab is assigned code L04AA36, which corresponds to antineoplastic and immunomodulating agents (L), immunosuppressant (L04), selective immunosuppressant (L04AA). The active substance rituximab is assigned code L01FA01, which corresponds to antineoplastic and immunomodulating agents (L), antineoplastic agent (L01), CD20 inhibitor (L01FA).

TABLE 1
ATC level 1 groups.

| Code | Description |
|---|---|
| A | Alimentary tract and metabolism |
| B | Blood and blood forming organs |
| C | Cardiovascular system |
| D | Dermatologicals |
| G | Genito-urinary system and sex hormones |
| H | Systemic hormonal preparations, excluding sex hormones and |
| | insulins |
| J | Anti-infectives for systemic use |
| L | Antineoplastic and immunomodulating agents |
| M | Musculo-skeletal system |
| N | Nervous system |
| P | Antiparasitic products, insecticides and repellents |
| R | Respiratory system |
| S | Sensory organs |
| V | Various |

Two different features were derived from ATC codes. The first feature was a similarity between complete ATC codes, where each ATC code is represented by all possible subsets of the ATC code (e.g. ATC code L04AA36 is represented by the set [L, L0, L04, L04A, L04AA, L04AA3, L04AA36] and ATC code L01FA01 is represented by the set [L, L0, L01, L01F, L01FA, L01FA0, L01FA01]-note that the last number is always unique to a drug and can therefore be included or excluded from this representation; in the present examples it was included). This representation was believed to capture more information in terms of similarity of ATC codes than a comparison of the whole code or level to level. Any similarity metric that can be used to compare discrete sets as described above may be used, such as e.g. the Jaccard or Dice similarities. In the present examples the inventors used the Jaccard similarity:

$$J = \frac{\vert X ∩ Y \vert}{\vert X ⋃ Y \vert} \qquad ( 17 )$$

In the example above, ocrelizumab and rituximab are assigned a similarity of 0.17 (the intersection of the two ATC representations is 2 elements (L and L0), and the union is 12 elements (L, L0, L04, L04A, L04AA, L04AA3, L04AA36, L01, L01F, L01FA, L01FA0, L01FA01), such that the Jaccard similarity is 2/12=0.167, rounded to 0.17).

The second feature was a feature encoded as a binary vector that assigns a 0 or 1 value to each of the 105 possible combinations of the 14 classes in the first ATC level. In other words, this feature indicates which of the first level classes the two drugs fall in. In the example above, ocrelizumab and rituximab are both in class L and therefore the corresponding feature is a vector comprising 0s for all classes apart from “L_L”. In other words, the first level of each drug in a pair were one hot encoded to produce a binary vector.

Imputation and data pre-processing. Various pre-processing pipelines were evaluated. Missing data was imputed in all cases using KNN (k-nearest neighbours) with k=10. This imputes a missing feature as the average across the 10 most similar drugs. The data was then: (i) used as is (labelled “rec_impute”), (ii) scaled and normalized (labelled “rec_impute_norm”), (iii) scaled, normalized and subject to PCA (principal component analysis; labelled “rec_impute_nrom_pca”), or (iv) scaled, normalized and subject to partial least square (PLS; labelled “rec_impute_norm_pls”). In the present examples the imputed, scaled and normalised data was used (labelled as “rec_impute_norm”. This is because performing PCA or PLS was not found to improve the model much in the present examples, and as these methods are computationally intensive it was more efficient to use rec_impute_norm for inference. Scaling and normalisation were always performed separately for each predictive feature. These steps can help resolve potential issues with the data to avoid some features dominating the predictions. In the present case as all features were between 0 and 1, scaling and normalising showed a moderate improvement but as it is computationally efficient and safe to apply this step, the step was maintained. The PCA and PLS were used only for the non-binary features. The number of non-binary features was reduced to 5 (top 5 principal components or top 5 latent variables of the PLS) using these approaches, which obtain summarised predictive variables that are linear combinations of the original variables.

Model architecture and training. A binary predictive XGBoost algorithm was trained on this data to predict synergy. The implementation in the R package xgboost was used (cran.r-project.org/web/packages/xgboost/index.html), via the package parsnip (parsnip.tidymodels.org/; cran.r-project.org/web/packages/parsnip/index.html). Independent features used for prediction included molecular structure, mode of action, side effects, pathway, disease association, protein interaction, network, and drug indication. As explained above, the model was trained on 80% of the data (excluding previous MS disease-modifying therapy combinations) and tested on 20% unseen data (FIG. 4, Step 3). Final tuning parameters were found by training the model by randomly dividing the data into 10 partitions (i.e. 10-fold cross-validation). The final model had 1237 trees, a depth of 6 and mtry (number of randomly selected predictors at each split) of 76. All of these hyperparameters were tuned using a grid search (as described in www.tmwr.org/grid-search) which tries a range of different parameters and uses the one with best performance. As the data used for training and testing is very unbalanced (very few positive examples compared to the number of negative examples), a threshold for calling predictions as synergistic was chosen as 0.29 (i.e. predicted probabilities >0.29 are called synergistic), based on the F1 score. The f1 score measures prediction accuracy by combining precision and recall as F1=(2*precision*recall)/(precision+recall) where precision—also known as specificity—is the rate of true positive predictions amongst positive predictions (TP/(TP+FP), where TP are true positives and FP are false positives), and recall—also known as sensitivity—is the rate of true positive predictions amongst real positives (TP/(TP+FN), where FN are false negatives). High F1 scores indicate both good precision and recall (i.e. the metric aims to maximise both of these competing objectives). The balanced accuracy (average of recall and precision) was also computed for all models. Feature selection was performed using recursive feature elimination as implemented in the R package caret (cran.r-project.org/web/packages/caret/index.html; topepo.github.io/caret/recursive-feature-elimination.html#rfe). Feature importance analysis was performed using DALEX (ema.drwhy.ai/featurelmportance.html) and the vip package (cran.r-project.org/web/packages/vip/index.html). Models were further evaluated by Bayesian analysis to compare models using rresampling statistics using package tidyposterior (cran.r-project.org/web/packages/tidyposterior/index.html), and by obtaining calibration curves using package probably as described in www.tidyverse.org/blog/2022/11/model-calibration.

Alternative model architectures. A variety of alternative architectures were also used, and each model was trained as described above. The tested model architectures included: a decision tree classifier (an ensemble of 1237 trees with a depth of 6 and mtry (randomly selected predictors) of 76), a random forest classifier, a logistic regression model, a multivariate adaptive regression spline (MARS), a regularized discriminant analysis (RDA), a multilayer perceptron (MLP), and a support vector machine (SVM) with a radial basis function (RBF) kernel. All models were accessed via the parsnip R package (cran.r-project.org/web/packages/parsnip/index.html), which accesses the following R packages: for the decision tree: rpart (cran.r-project.org/web/packages/rpart/rpart.pdf); for the random forest: ranger (cran.r-project.org/web/packages/ranger/ranger.pdf); for the logistic regression: glm function in stats package (stat.ethz.ch/R-manual/R-devel/library/stats/html/glm.html); for the MARS: earth (cran.r-project.org/web/packages/earth/index.html); for the RDA: klaR (cran.r-project.org/web/packages/klaR/index.html); for the MLP: nnet (cran.r-project.org/web/packages/nnet/index.html); for the SVM: kernlab (cran.r-project.org/web/packages/kernlab/index.html).

Validation on MS disease modifying drugs. The model was applied to 33,313 pairs of drugs, including combinations of 17 existing disease-modifying therapies for MS and 7,678 approved or investigational medications for various diseases (FIG. 4, step 4). These were selected as any DMT for MS in phase 1 or above in OpenTargets (www.opentargets.org), which uses ChEMBL, recorded in These were filtered from the total number of possible combinations to exclude combinations with more than 3 missing values of predictive features. The disease modifying therapies included: alemtuzumab (CHEMBL1201587), cladribine (CHEMBL1619), ocrelizumab (CHEMBL2108041), natalizumab (CHEMBL1201607), ofatumumab (CHEMBL1201836), fingolimod (CHEMBL314854), ponesimod (CHEMBL1096146), interferon-beta-1b (CHEMBL1201563), interferon-beta-1a (CHEMBL1201562), mitoxantrone (CHEMBL58), Siponimod (CHEMBL2336071), dimethylfumarate (CHEMBL2107333), glatiramer acetate (CHEMBL1201507), teriflunomide (CHEMBL973), diroximel fulmarate (CHEMBL3989944), azathioprine (CHEMBL1542), and ozanimod (CHEMBL3707247).

### Results

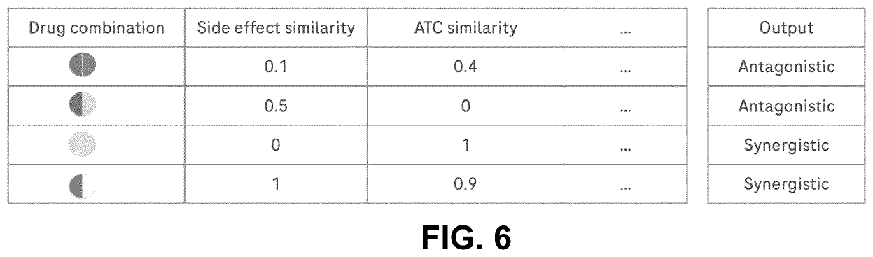

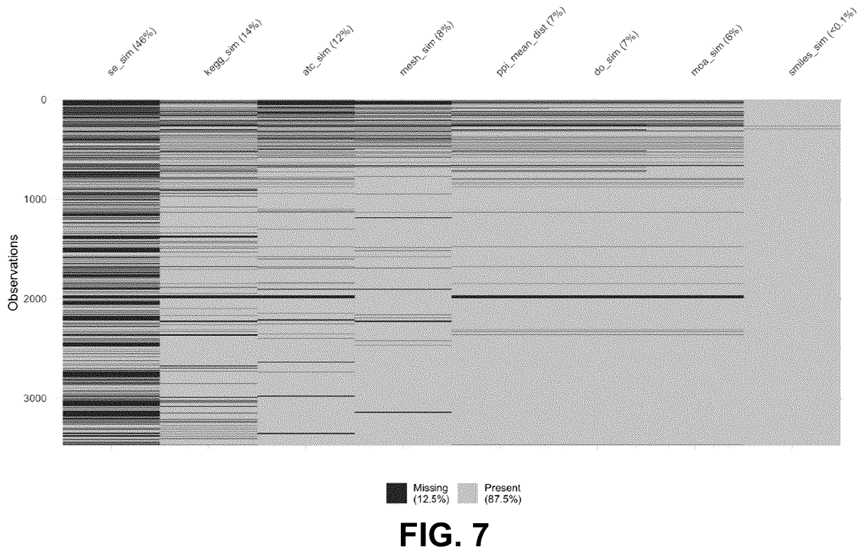

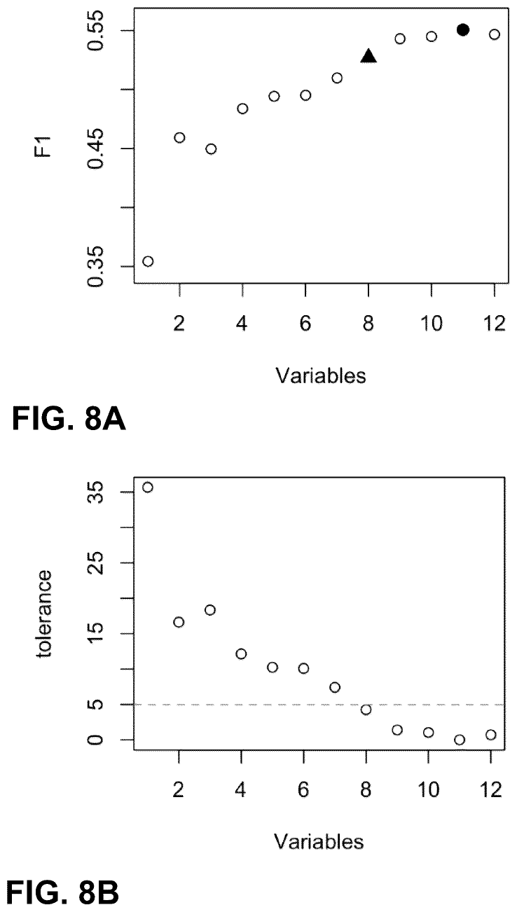

The structure of the data used for prediction is illustrated schematically on FIG. 6. For each drug combination for which clinical trial data was available, all of the candidate predictive features described above were computed. Each drug combination was further labelled as “synergistic” or “antagonistic”. The data was then analysed to determine the extent of missing values in the data. This is illustrated on FIG. 7 which also shows the overall proportion of missing values (12.5%) as well as the proportion of missing values per feature (from left to right: side effect similarity, kegg pathway similarity, ATC similarity, MESH similarity, closeness on PPI, disease ontology similarity, mode of action similarity and structural similarity (labelled as “smiles similarity”). This indicated that there was likely a need for data imputation in this dataset, as removing any entry with missing data would remove too much of the dataset. The data on FIG. 7 show the overall pattern of missingness, illustrating the challenges in dealing with real world data, as well as the potential for large benefits associated with data engineering and cleaning. For example, the data show that the side effects feature has a lot of missing values. This is because usually approved drugs (~2000 drugs) have their side effect labels. Therefore, the inventors tested 2 approaches: imputing this missing data or using a model without this feature. The later resulted in a model with lower performance, so the feature was kept and missing data was imputed. Nevertheless, to limit the impact of missing data and imputation, the inventors decided to remove all rows (i.e. any drug combination) which had >2 missing values. They then imputed the missing values as part of the preprocessing (rec_impute_norm) as described above. Then, feature selection was performed by recursive feature elimination, i.e. starting from the full set of variables tested, models were tested with progressively fewer variables and the F1 score was computed. Each time, the variable that has the minimal effect on the performance is removed. The results of this are shown on FIGS. 8A-8B, which shows results as the average of 10-fold cross validation (FIG. 8A shows the average F1 score and FIG. 8B shows the average tolerance %, i.e. the percentage of error accepted). A 5% tolerance threshold was used, accepting a model with F1 score up to 5% below that of the best model (indicated as a dashed line on FIG. 8B, leading to selection of the 8 features model with F1 score highlighted as the solid triangle on FIG. 8A, which is the smallest model with performance within 5% of the best model, indicated as a solid dot on FIG. 8A). Based on this, the inventors decided to remove the following predictive features: target similarity, pathway similarity based on Gene ontology and Reactome (maintaining pathway similarity based on Kegg annotations), and co-expression. The following features were therefore maintained in the final models: molecular structure similarity, mode of action similarity, side effects similarity, pathway similarity (Kegg annotations), disease association similarity, closeness on protein-protein interaction network, MESH similarity, and ATC similarity (both whole code similarity and first level binary similarity).

These features were used to train an XGBoost model which achieved a specificity of 94% and sensitivity of 70% in predicting synergistic combinations when evaluated on unseen data (20% hold out test data).

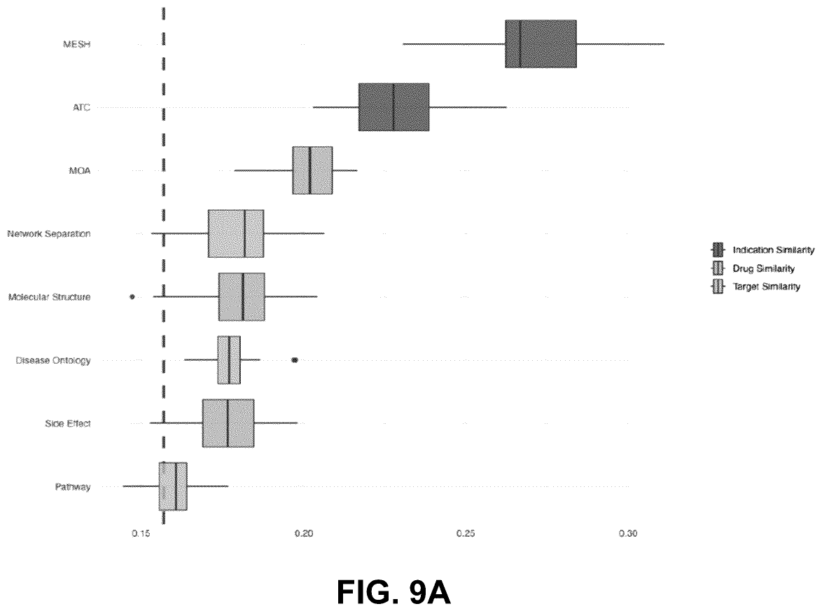

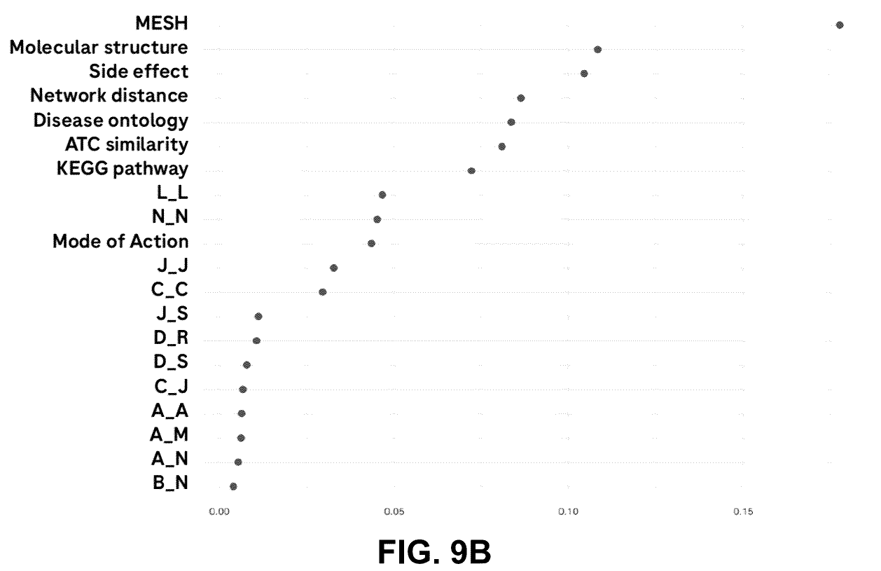

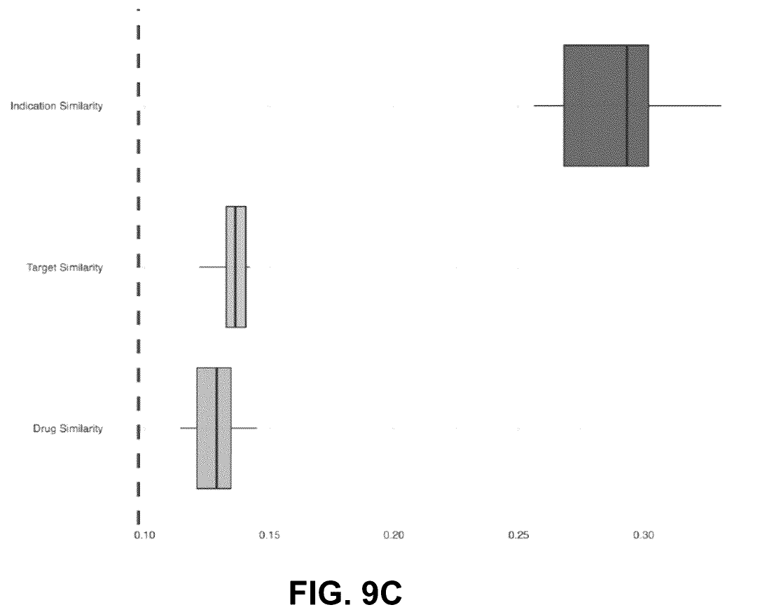

FIGS. 9A-9C show the results of a feature importance analysis. First, the relative importance of the predictive features in this model was investigated by randomly permuting the variables in the test data and calculating the mean of (1-AUC) obtained by applying the model trained to 10 random perturbations of each of the indicated features (i.e. shuffling the values of a feature, predicting from the model and measuring how much worse the model fits the data compared to before shuffling). The results of this are shown on FIG. 9A, where the black line is the median across the 10 perturbations, the box shows the interquartile range and the whiskers extend to the minimum and maximum values. For this analysis the binary ATC features were removed for computational efficiency. FIG. 9A shows that the indication similarity features (MESH and ATC) were most important to the model's prediction, followed by the mode of action similarity. The dashed line shows the performance of the full model, so the further a distribution of (1-AUC) values is from this line, the more important the feature (i.e. the model loses more performance when the feature is disturbed). FIG. 9B shows the results of an investigation of feature importance considering the ATC first level features as individual features. For this analysis all features were used (8 features+binary ATC codes) and no permutation was performed as that was too computationally heavy when including all binary features, so the variance-based approach described in Greenwell et al. 2018 as implemented in R package vip was used. This shows that again the MESH similarity feature is most important to the predictions, but also shows that the importance of the ACT similarity feature importance is actually separated between the ATC similarity (whole code similarity) and the first level binary similarity in particular for some combinations (e.g. if a pair of drug is in the L_L, N_N, J_J, or C_C categories then this is very informative of whether the drugs will synergise, whereas other categories are less informative). Some of these first level binary similarity categories (in particular, L_L and N_N) are even more informative than the mode of action feature. FIG. 9C shows results of the same analysis as as above on FIG. 9A but grouping the features by category (i.e. indication similarity, target similarity, and drug similarity). Features were grouped into each of these groups and perturbed together, then model performance was quantified as (1-AUC) across 10 permutations as described above. This shows that the indication similarity features (combining MESH similarity and both types of ATC similarity) are by far the most important to the predictions made by the model.

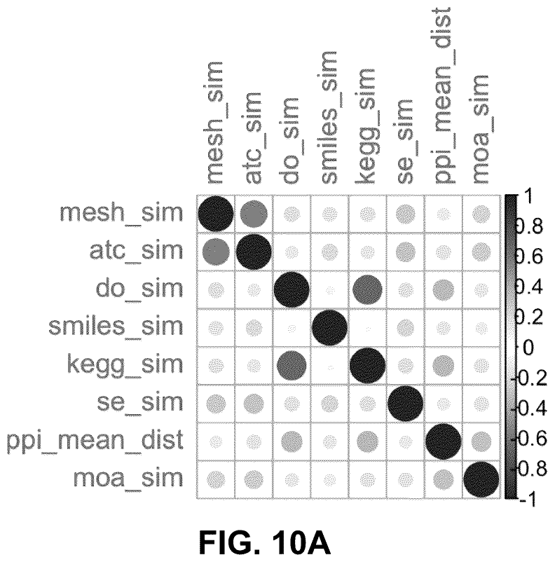

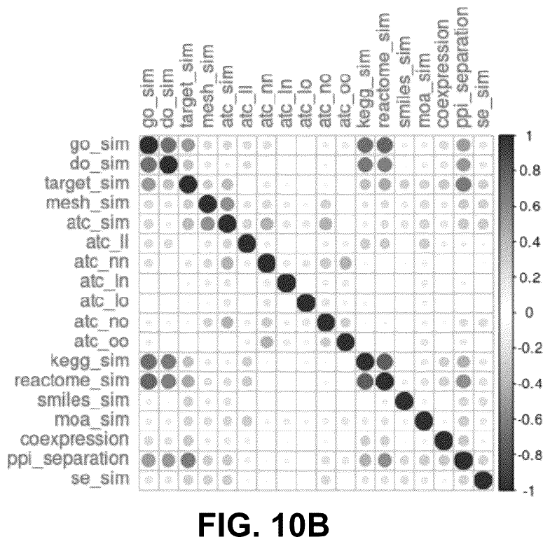

FIGS. 10A-10B show the Pearson correlation between the features of the final predictive model, across the training and test dataset for the 8 features in the 8 features model (A) and for all features initially investigated (B). For the ATC similarity only the complete code similarity metric is used to produce the plot on FIG. 10A. The colour intensity and size of the dots indicates the correlation between the variables, and the colour (red or blue) indicates the sign of the correlation. The data on FIG. 10A show that the ATC and MESH similarities are highly correlated, as are the disease ontology and pathway similarity (kegg_sim), as expected. However, the data on FIG. 9A indicate that at least when it comes to ATC and MESH, the information captured is not fully redundant. FIG. 10B shows that kegg, gene ontology and Reactome-based pathway similarities are highly correlated (underlying the decision to only include one of them in the set of predictive features used, in the present examples the Kegg-based one, although any of these could have equally been used).

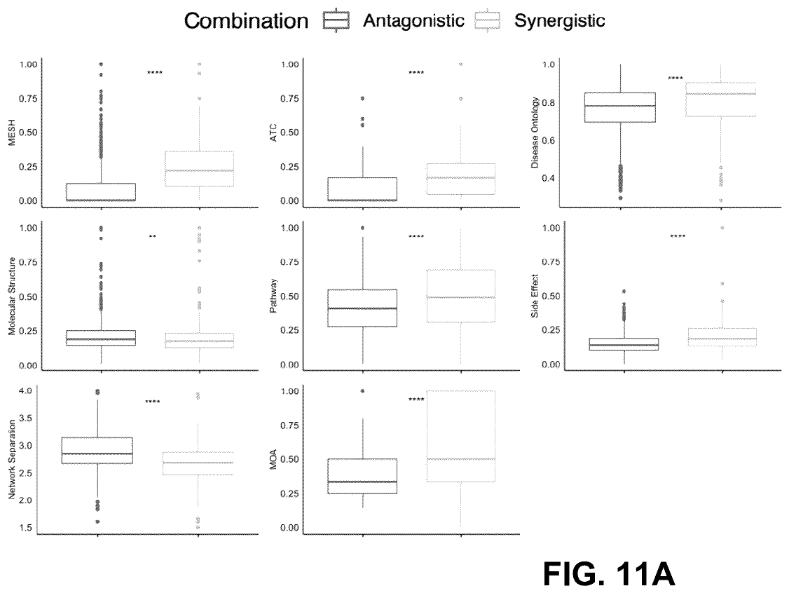

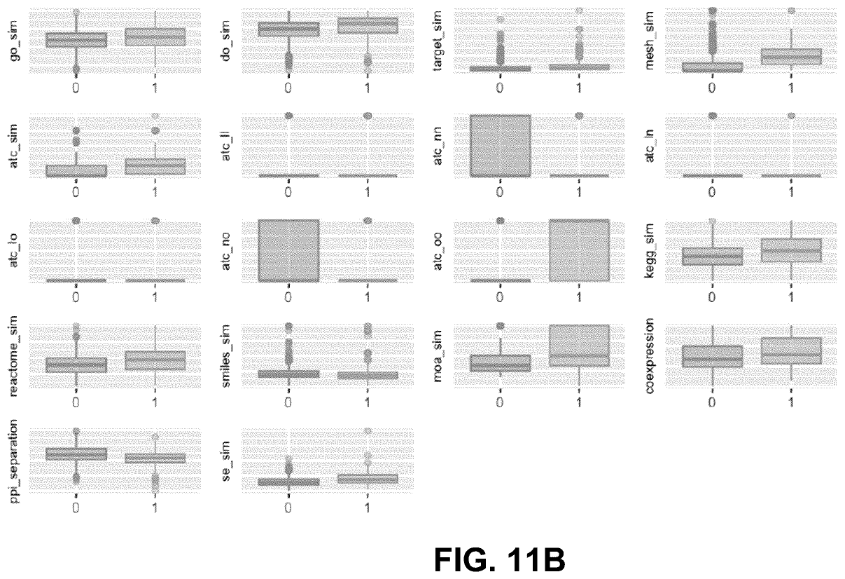

FIGS. 11A-11B show the distribution of each of the predictive features in the training data, separated by class of combinations (Antagonistic on the left, synergistic on the right). FIG. 11A shows the results for the variables included in the final models. All differences were found to be statistically significant (test: Wilcoxon test, **: p<=0.01; ***: p<=0.001; ****: p<=0.0001). The data shows that the importance of predictive features is not necessarily related to the magnitude of the differences between the two distributions. For example, the mode of action similarity distributions are more different from each other than the MESH and ATC similarity (whole code similarity) distributions owing to the much wider spread of values in the synergistic classes. FIG. 11B shows the same data for all variables initially investigated. The data shows that the features that were removed (coexpression, go_sim and reactome_sim) either have small differences between the categories or have distributions that are similar to other predictive features.

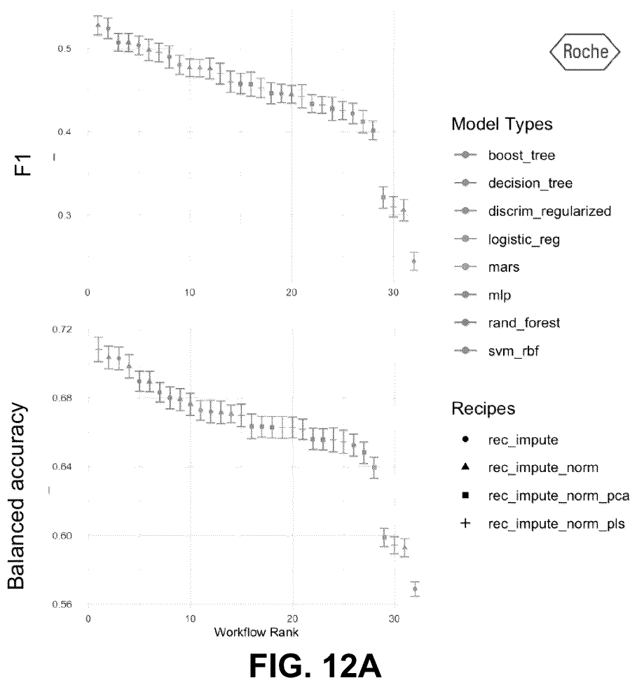

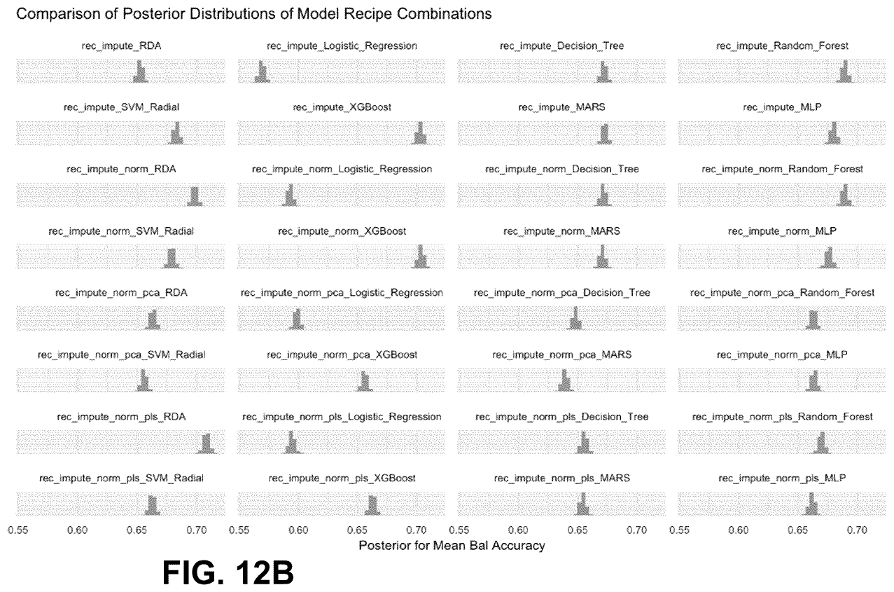

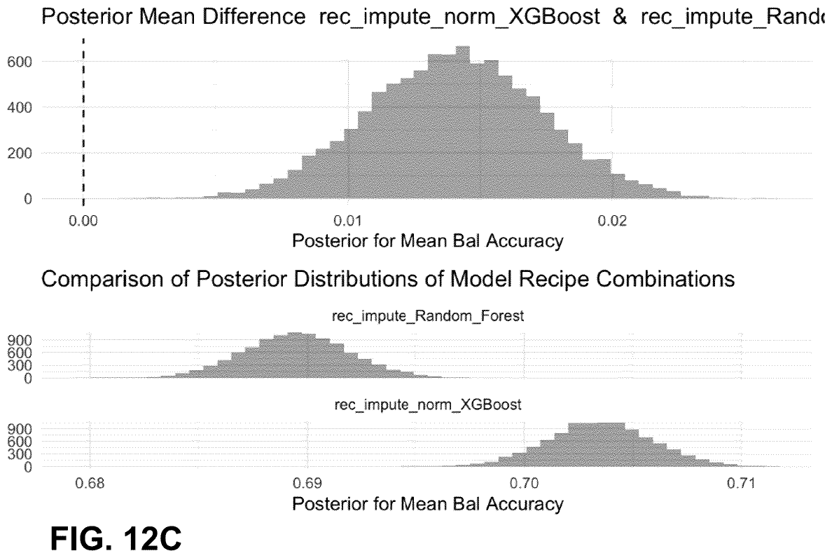

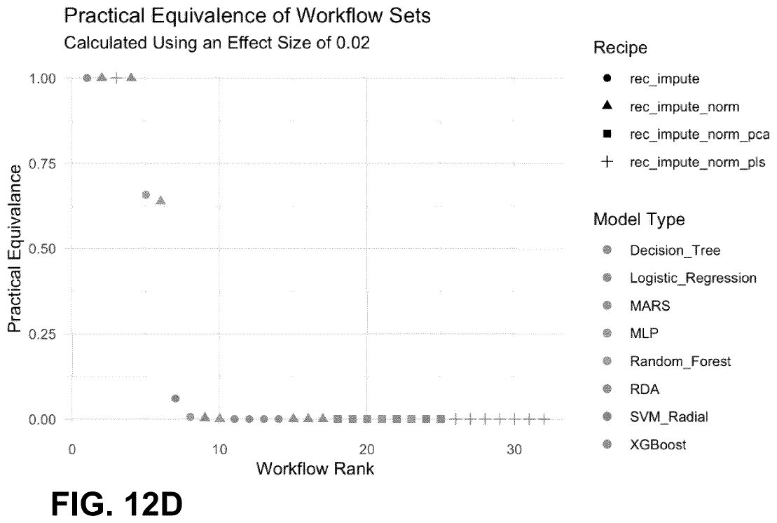

FIGS. 12A-12D show the results of studies investigating the effect of the choice of model and data pre-processing method. FIG. 12A shows the effect of model choice and data preprocessing on F1 scores and balanced accuracy. F1 and balanced accuracy were used as performance metrics because the dataset was very unbalanced (including many more antagonistic pairs than synergistic pairs). For each model and data pre-processing method tried, the F1 accuracy is shown (top), ranked by decreasing F1, and the balanced accuracy is shown (bottom), ranked by decreasing balanced accuracy. The data shows that logistic regression performed more poorly than all other models, while boosted trees performed particularly well. However, all models apart from the logistic regression model showed good performance. Non-linear models tended to perform better than linear models (logistic regression, regularised discriminant analysis). All data preprocessing approaches were suitable but normalised data performed slightly better in general, and dimensionality reduction by PCA/PLS did not appear to be beneficial. FIG. 12B shows the posterior distributions of mean balanced accuracy associated with each of the indicated combinations of models and pre-processing methods. This compares multiple models by resampling, evaluating performance (in this case balanced accuracy) of submodels on cross-validation resamples. Using these observations, a Bayesian method is used to calculate the posterior distribution of the balanced accuracy for each model. This confirms that the models using normalised data have higher performance than the corresponding models without normalisation, and shows the XGBoost models as clearly performing better than all other models, followed by random forest and multi-layer perceptrons. FIG. 12C more closely compares the random forest and XGboost models in terms of posterior distributions of mean balanced accuracy. The top panel shows the difference of mean balanced accuracy between the XGboost model on normalised data and between the random forest on imputed but not normalised data (note that with the random forest model there was no strong difference associated with the use of normalised data, see FIG. 12B). The bottom panels show zoomed in versions of the respective posterior distributions of mean balanced accuracy associated with these models shown on FIG. 12B. This shows that the XGBoost model does perform slightly better than the random forest model. FIG. 12D shows the proportion of models with an effect size of 2%, where effect size is the amount of difference in balanced accuracy that we are willing to accept. This again shows that the XGBoost model has best performance, followed by RDA and random forest, and that normalisation has a small effect on performance of these models.

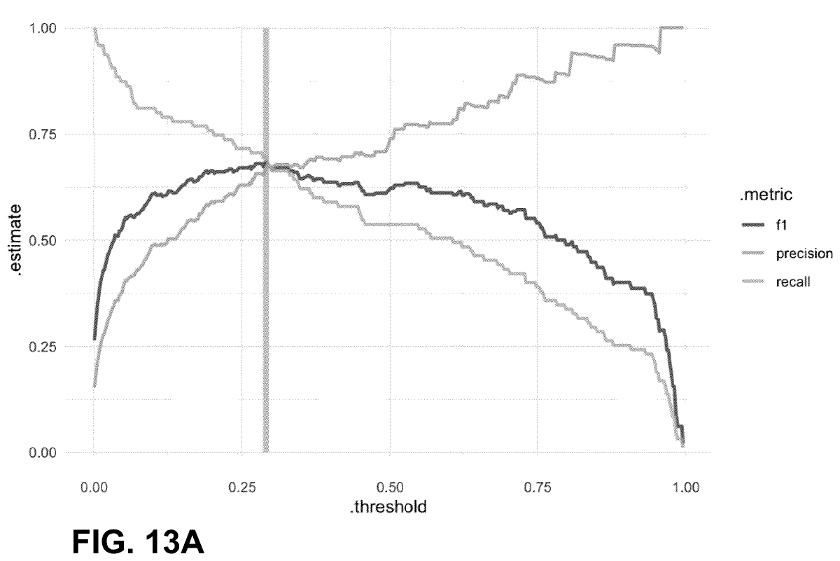

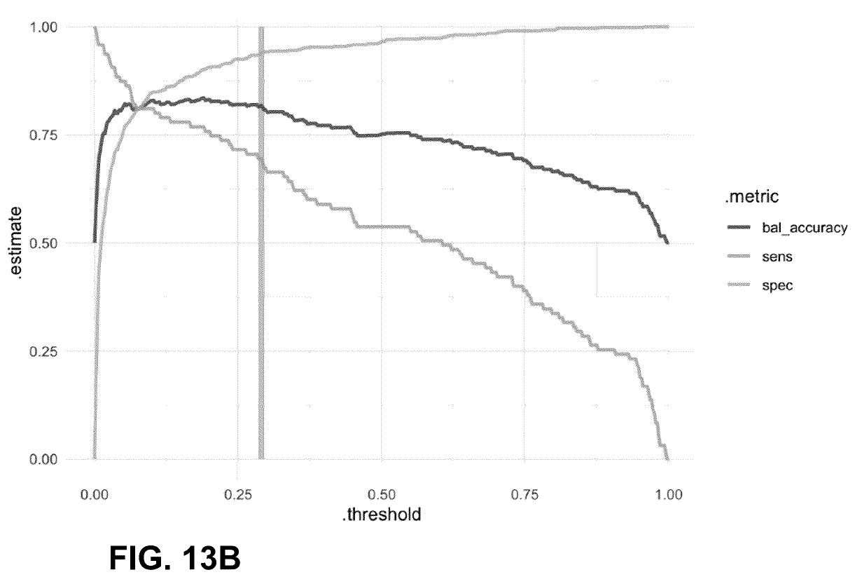

FIGS. 13A-13B show the results of threshold selection and optimisation processes used to obtain the final XGboost model. The data on FIG. 13A shows the F1 score, precision (increasing data series) and recall (decreasing data series) obtained by XGBoost models as the threshold for calling predictions as “positive” (synergistic) increases, for the training data. This shows that the threshold value of 0.29 was found to maximise the F1 score. The F1 score was chosen as a performance metric because the dataset was imbalanced. However, the same approach can be applied to maximise any other metric such as precision or recall. FIG. 13B shows the balanced accuracy, sensitivity (decreasing data series) and specificity (increasing data series) obtained by XGBoost models as the F1 threshold increases (same models as on FIG. 13B, but evaluating on different performance metrics). This figure shows that even though we maximize F1 score (not the bal_accuracy), the value of bal_accuracy with the threshold of 0.29 is close to its maximum. So by choosing 0.29 we have high F1 and bal_accuracy.

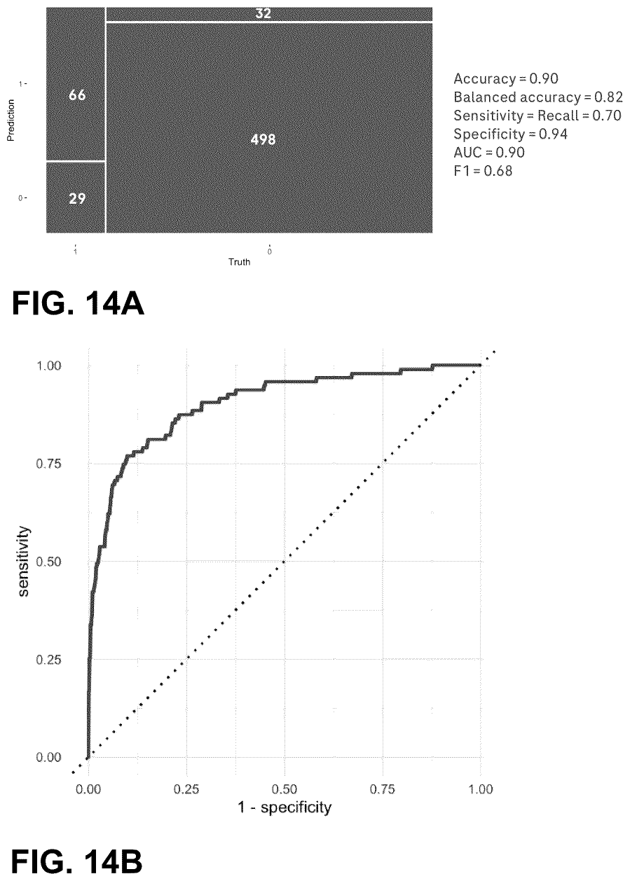

FIG. 14A shows a confusion matrix obtained by evaluating the trained XGBoost model (trained with the above features and selected based on F1 score=0.29 threshold as described above) on the hold out test data. This shows that the mode achieved a specificity of 94% and sensitivity of 70% in predicting synergistic combinations when evaluated on unseen data (20% hold out test data). This is exceptionally good performance. The classification accuracy was 90%. This is higher than e.g. the best model described in Ji et al. 2019, which is based on a similar architecture but uses different predictive features (accuracy 78.7%). FIG. 14B shows the associated receiver operator characteristic (ROC) curve, again showing very high prediction performance.

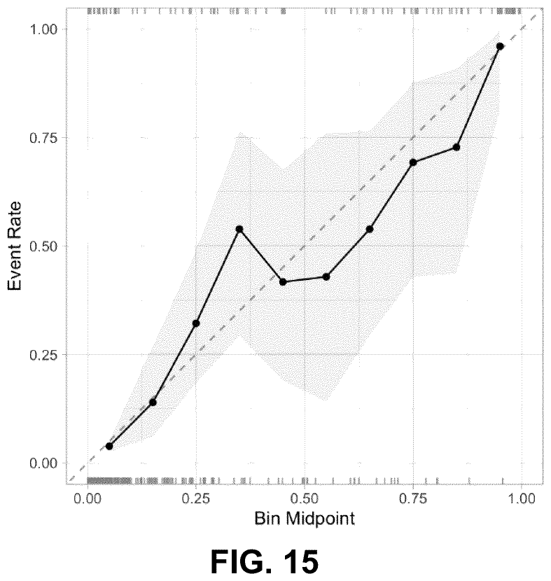

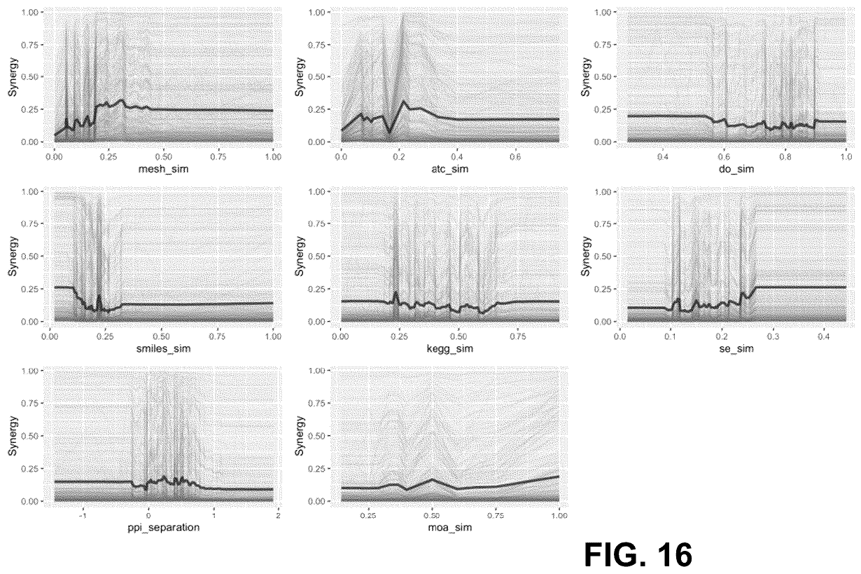

FIG. 15 is a calibration curve showing the observed numbers of synergistic pairs (event rate) for each of a plurality of bins of predicted probabilities from the XGboost model (x axis indicating the midpoint of the bins). The idea behind a calibration plot is that if we group the predictions based on their probability, then we should see a percentage of events that match such probability. For example, if we collect a group of the predictions whose probabilities are estimated to be about 10%, then we should expect that about 10% of the those in the group to indeed be events. The plots shown here can be used as diagnostics to see if the predictions of the model are consistent with the observed event rates. In this plot, event rate is the ratio of synergistic to antagonistic. The dashed lines on top shows the synergistic combination probabilities, and dashed lines on bottom shows the antagonistic probabilities. Usually the challenging part is the tails (close to 0 or 1) but the data shows that the model is performing well (close to x=y line) The plots shows that the prediction of the model are consistent with the observed event rates, indicating that the model performs well.

FIG. 16 shows partial dependence profiles for pairs of predictive features. Partial dependence profiles show how the expected value of a model prediction (synergy) changes as a function of a feature (mesh_sim, atc_sim, . . . ) Here each gray line corresponds to a row in test dataset (i.e. a single drug combination, its features, and its synergy). The blue line corresponds to the average of all the gray lines. This plot shows the overall behaviours of output when we change each feature. For example, when we increase the mesh_sim score from 0 to 1, the synergy probability increases. This makes biological sense. Thus, this plot is a diagnostic plot to ensure that the predictive features of the model behave in a way that makes sense biologically-which is the case here.

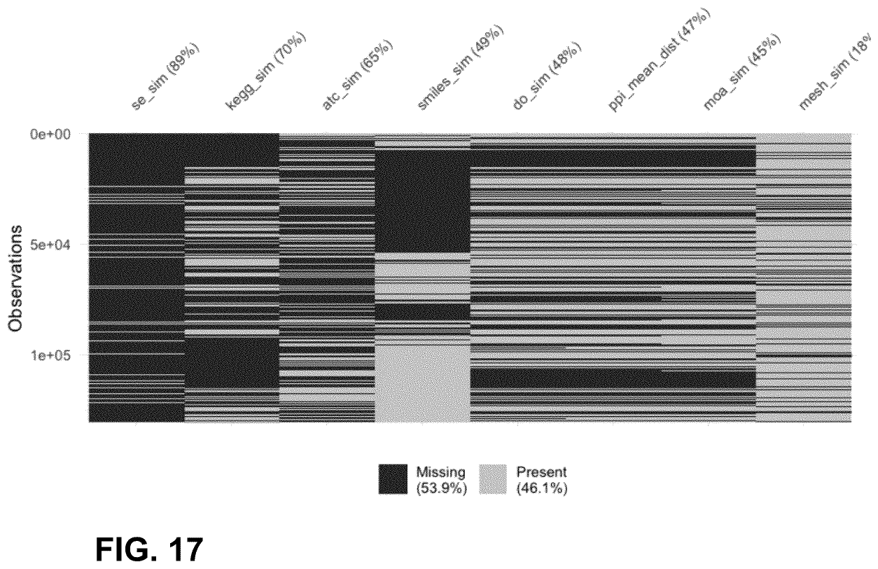

Next, the trained model was applied to combinations of 17 validated disease modifying therapies (DMTs) for the treatment of MS, together with drugs currently in phase 2 or above clinical trials (7,678 investigational drugs, leading to approximately 130,000 combinations). FIG. 17 shows the missing values for this data. Each row is a drug combination, and black indicates a missing value. This shows that this data contains a higher proportion of missing values, primarily in relation to side effects, kegg and ATC similarities. This is not unexpected as many of these drugs may not yet have fully characterised side effects and an ATC code, and some of the investigational drugs do not even have a known target (leading to a larger proportion of missing values for features like keg pathway similarity which rely on known targets). However, the data also shows that the mesh similarity feature has the lowest % of missing values. This is particularly significant considering that the feature was found to be highly predictive. Therefore, this indicates that predictions from the present model are likely to improve even more on prior art models for disease areas such as MS where few marketed DMTs are available, since the predictions can make use of real, highly informative data rather than imputation for these drug combinations.

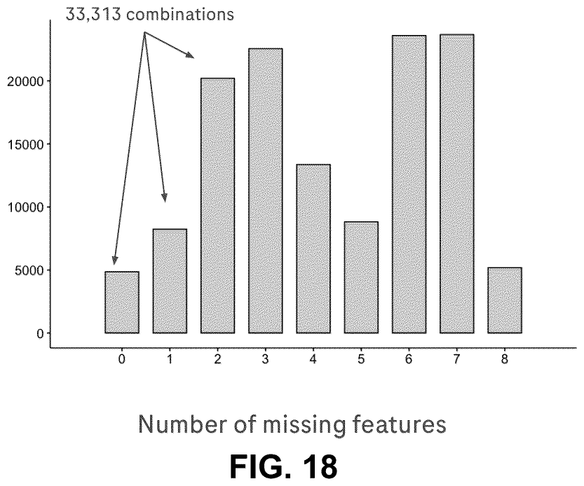

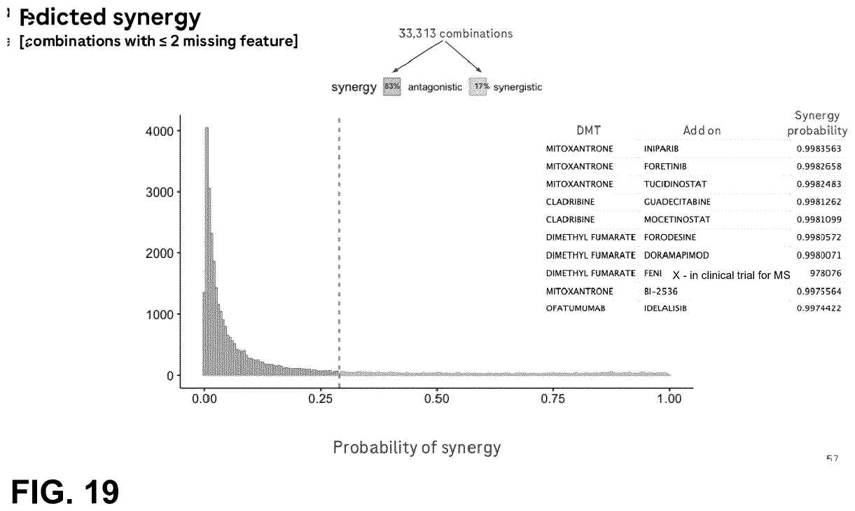

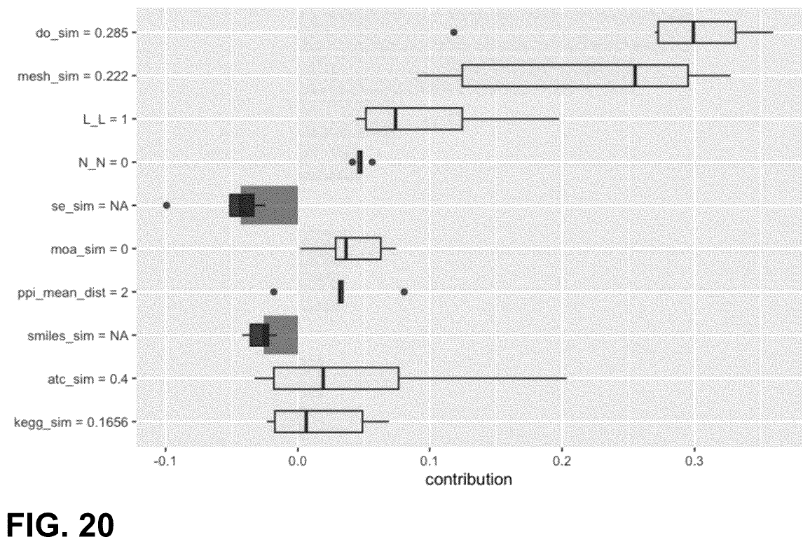

FIG. 18 shows a histogram of the distribution of the number of missing features per drug combination in the MS drug combination data. In view of the very large amount of missing data in this dataset, further analysis was focussed on the 33,313 combinations for which the maximum number of missing features was 2 (i.e. 0, 1 or 2 missing features). The resulting predictions are shown as a histogram on FIG. 19. Of the 33,313 combinations, 17% were predicted to be synergistic when applying the optimised probability threshold of 0.29. The top predicted pairs are listed in the inset panel together with the MS disease modifying therapy (DMT) in the first column, the additional therapy in the second column and the probability predicted by the model in the third column. This was also repeated for a particular single DMTs of interest, i.e. the top 3 predictions were extracted and the scientific literature was searched for any evidence of an involvement of the additional therapies in MS or MS related pathways. A synergistic effect for all top 3 predictions was supported by literature, either with direct evidence of synergy (in individual cases where the two therapies were administered concomitantly for unrelated conditions, or at the preclinical level, as all these combinations are entirely new ones that have not yet been made the object of clinical trials). The feature importance for one of these top 3 predictions are shown on FIG. 20, together with the actual values of the features for this combination (y axis labels).

This plot shows why this combination gets the probability that is assigned to it (which is 0.87). The x-axis shows the values of the predictive features for this combination (do_sim=0.285, mesh_sim=0.222, . . . ). The x axis shows how much each feature contributes towards the predicted probability. For example, do_sim contributes 0.3 out of 0.87. This was obtained by computing how contributions attributed to individual features change the mean model's prediction for a particular observation as Shapley additive explanations. The boxplots show the distribution of contributions across all orderings used to compute the Shapley attributions. This plot shows that for this particular combination, the disease ontology and mesh similarities are the largest contributors to the probability obtained.

### CONCLUSION

This work shows that machine learning provides a valuable tool for understanding drug characteristics and successful combinations. This study designed high-performance models that identified potentially successful combinations for further assessment in experimental trials. It is the first model validated specifically for predicting drug combinations in neurological diseases like MS, where combination trials for MS are kept as unseen data and used for validation. The model not only identified new combinations but also accurately predicted the results of all the previous combination trials of MS that have been tried based on biological evidence and expert opinions. Further curation can select the most promising predicted synergistic candidates aligned with specific development goals.

This study demonstrates the potential of machine learning in discovering effective drug combinations for MS treatment. It addresses the gaps in available therapies and holds promise for enhancing MS management. Further exploration and validation in experimental trials can be applied to evaluate the identified combinations and advance MS treatment options.

Further, the data demonstrates the power of a number of new ways of characterising drug combinations for the purpose of predicting the likelihood of synergy. A first newly developed feature of high importance was the drug target(s) MESH similarity. As shown on FIG. 9A, this new feature contributes extremely valuable information to the synergy prediction. A second newly developed feature of importance was the ATC first level combination feature. As shown on FIG. 9B, this approach to characterising pairs of drugs contributes information that is not captured by a simple ATC similarity metric, and at least some of these ATC pair categories were extremely informative in making predictions. Together, these features make up the most important part of the predictive model, as shown on FIG. 9C (where the indication similarity features importance is the combination of the importance of the MESH similarity, ATC similarity and ATC binary categories variables). A third feature of importance is the disease ontology similarity metric, which as shown on FIGS. 9A-9B was more important to the predictions made than side effects or even ATC similarity if considered separately from the ATC binary categories variables. Importantly, both the MESH similarity and disease ontology similarity rely on the availability of annotations of the drug targets, and as such are available even for drugs that have not yet been assigned an ATC or that do not have side effects registered in the FDA Orange book. This means that values for these predictor features are available for a much broader range of drugs, enhancing the applicability of the model as well as the amount of accurate available training data. Therefore, it is believed that the use of these the 3 features above, each individually, and collectively, underlie the benefits of the present models including its very high prediction performance.

### REFERENCES

All documents mentioned in this specification are incorporated herein by reference in their entirety.
Revilla Sancho L (2023). BioCor: Functional similarities. bioconductor.org/packages/BioCor, Ilrs.github.io/BioCor/.
Guha Rajarshi, fingerprint: Functions to Operate on Binary Fingerprint Data, package version 3.5.7., published 2018 Jan. 7, CRAN.R-project.org/package=fingerprint
Csárdi G, Nepusz T, Traag V, Horvát S, Zanini F, Noom D, Müller K (2023). igraph for R: R interface of the igraph library for graph theory and network analysis doi: 10.5281/zenodo.7682609, R package version 1.5.1, CRAN.R-project.org/package=igraph.
G Yu, L G Wang, G R Yan, Q Y He. DOSE: an R/Bioconductor package for Disease Ontology Semantic and Enrichment analysis. Bioinformatics 2015, 31 (4):608-609. dx.doi.org/10.1093/bioinformatics/btu684.
Yu G (2018). “Using meshes for MeSH term enrichment and semantic analyses.” Bioinformatics, 34(21), 3766-3767. doi: 10.1093/bioinformatics/bty410.
Schriml L M et al. Disease Ontology: a backbone for disease semantic integration Nucleic Acids Res. 2012 40 D940 D946
Peng K, Xu W, Zheng J, Huang K, Wang H, Tong J, Lin Z, Liu J, Cheng W, Fu D, Du P, Kibbe W A, Lin S M, Xia T. The Disease and Gene Annotations (DGA): an annotation resource for human disease. Nucleic Acids Res. 2013 January; 41 (Database issue): D553-60.
Wang J Z, Du Z, Payattakool R, Yu P S, Chen C F. A new method to measure the semantic similarity of G O terms. Bioinformatics. 2007 May 15; 23(10):1274-81.
Resnik P. Semantic similarity in a taxonomy: an information-based measure and its application to problems of ambiguity in natural language, J. Artificial Intelligence Res., 1999, vol. 11 (pg. 95-130)
Jiang J, Conrath D. Semantic Similarity Based on Corpus Statistics and Lexical Taxonomy, 1997tenth International Conference on Research on Computational Linguistics (ROCLING X) Taiwan
Lin D. An information-theoretic definition of similarity, Semantic Similarity Based on Corpus Statistics and Lexical Taxonomy, fifteenth International Conference on Machine Learning, 1998 (pg. 296-304)
Schlicker, Andreas, Francisco S Domingues, Jörg Rahnenführer, and Thomas Lengauer. 2006. “A New Measure for Functional Similarity of Gene Products Based on Gene Ontology.” BMC Bioinformatics 7:302.
Ying Tao, Lee Sam, Jianrong Li, Carol Friedman, Yves A. Lussier, Information theory applied to the sparse gene ontology annotation network to predict novel gene function, Bioinformatics, Volume 23, Issue 13, July 2007, Pages i529-i538
Rogers D, Hahn M. Extended-connectivity fingerprints. J Chem Inf Model. 2010 May 24; 50(5):742-54.
Ji X et al. Five-Feature model for developing the classifier for synergistic vs. antagonistic drug combinations built by XGBoost. Frontiers in Genetics (2019) 10:600.
Paltun B G, Kaski S., Mamitsuka H. Machine learning approaches for drug combination therapies. Briefings in Bioinformatics, 22(6), 2021, 1-16.
Nakazato T, Bono H, Matsuda H, Takagi T. Gendoo: functional profiling of gene and disease features using MeSH vocabulary. Nucleic Acids Res. 2009 July; 37 (Web Server issue): W166-9.
Maglott D, Ostell J, Pruitt K D, Tatusova T. Entrez Gene: gene-centered information at NCBI. Nucleic Acids Res. 2007 January; 35 (Database issue): D26-31.
FDA, 2023: U. S. Department of Health and Human Services Food and Drug Administration. Approved Drug Products with Therapeutic Equivalence Evaluations. 43^{rd} Edition, 2023, Products approved under section 505 of the Federal Food, Drug and Cosmetic Act, current through Dec. 31, 2022.
Liu H, Zhang W, Zou B, Wang J, Deng Y, Deng L. DrugCombDB: a comprehensive database of drug combinations toward the discovery of combinatorial therapy. Nucleic Acids Res. 2020 Jan. 8; 48(D1):D871-D881.
Wishart D S, Feunang Y D, Guo A C, Lo E J, Marcu A, Grant J R, Sajed T, Johnson D, Li C, Sayeeda Z, Assempour N, lynkkaran I, Liu Y, Maciejewski A, Gale N, Wilson A, Chin L, Cummings R, Le D, Pon A, Knox C, Wilson M. DrugBank 5.0: a major update to the DrugBank database for 2018. Nucleic Acids Res. 2017 Nov. 8.
Sharon L Freshour, Susanna Kiwala, Kelsy C Cotto, Adam C Coffman, Joshua F McMichael, Jonathan J Song, Malachi Griffith, Obi L Griffith, Alex H Wagner, Integration of the Drug-Gene Interaction Database (DGIdb 4.0) with open crowdsource efforts, Nucleic Acids Research, Volume 49, Issue D1, 8 Jan. 2021, Pages D1144-D1151.
Cao, D.-S. et al. (2015) Rcpi: R/Bioconductor package to generate various descriptors of proteins, compounds and their interactions. Bioinformatics, 31, 279-281.
Maggiora, G. M., Shanmugasundaram, V. (2011). Molecular Similarity Measures. In: Bajorath, J. (eds) Chemoinformatics and Computational Chemical Biology. Methods in Molecular Biology, vol 672. Humana Press, Totowa, N J.
Musnier, A.; Bourquard, T.; Vallet, A.; Mathias, L.; Bruneau, G.; Ayoub, M. A.; Travert, O.; Corde, Y.; Gallay, N.; Boulo, T.; et al. A New in Silico Antibody Similarity Measure Both Identifies Large Sets of Epitope Binders with Distinct CDRs and Accurately Predicts Off-Target Reactivity. Int. J. Mol. Sci. 2022, 23, 9765.
G Yu, F Li, Y Qin, X Bo*, Y Wu, S Wang*. GOSemSim: an R package for measuring semantic similarity among G O terms and gene products. Bioinformatics. 2010, 26(7):976-978.
Szklarczyk et al. Nucleic acids research 47.D1 (2018): D607-D613.2
Nakazato T, Bono H, Matsuda H, Takagi T. Gendoo: functional profiling of gene and disease features using MeSH vocabulary. Nucleic Acids Res. 2009 July; 37 (Web Server issue):W166-9.
Brandon M. Greenwell, Bradley C. Boehmke, Andrew J. McCarthy. A Simple and Effective Model-Based Variable Importance Measure. arXiv: 1805.04755v1 [stat.ML]. 12 May 2018.
Kanehisa M., Furumichi M., Tanabe M., Sato Y., Morishima K. KEGG: new perspectives on genomes, pathways, diseases and drugs. Nucleic Acids Res. 2017; 45: D353-D361.
Fabregat A., Sidiropoulos K., Garapati P., Gillespie M., Hausmann K., Haw R., Jassal B., Jupe S., Korninger F., Mckay S. et al., The reactome pathway Knowledgebase. Nucleic Acids Res. 2016; 44:D481-D487.
Caspi R., Billington R., Ferrer L., Foerster H., Fulcher C. A., Keseler I. M., Kothari A., Krummenacker M., Latendresse M., Mueller L. A. et al., The MetaCyc database of metabolic pathways and enzymes and the BioCyc collection of pathway/genome databases. Nucleic Acids Res. 2016; 44:D471-D480.
The Gene Ontology, C Expansion of the Gene Ontology knowledgebase and resources. Nucleic Acids Res. 2017; 45:D331-D338.

### EQUIVALENTS AND SCOPE

Unless context dictates otherwise, the descriptions and definitions of the features set out above are not limited to any particular aspect or embodiment of the invention and apply equally to all aspects and embodiments which are described.

“and/or” where used herein is to be taken as specific disclosure of each of the two specified features or components with or without the other. For example “A and/or B” is to be taken as specific disclosure of each of (i) A, (ii) B and (iii) A and B, just as if each is set out individually herein.

It must be noted that, as used in the specification and the appended claims, the singular forms “a,” “an,” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by the use of the antecedent “about” or “approximately”, it will be understood that the particular value forms another embodiment. The terms “about” or “approximately” in relation to a numerical value is optional and means for example +/−10%.

Throughout this specification, including the claims which follow, unless the context requires otherwise, the word “comprise” and “include”, and variations such as “comprises”, “comprising”, and “including” will be understood to imply the inclusion of a stated integer or step or group of integers or steps but not the exclusion of any other integer or step or group of integers or steps.

Other aspects and embodiments of the invention provide the aspects and embodiments described above with the term “comprising” replaced by the term “consisting of” or “consisting essentially of”, unless the context dictates otherwise.

The features disclosed in the foregoing description, or in the following claims, or in the accompanying drawings, expressed in their specific forms or in terms of a means for performing the disclosed function, or a method or process for obtaining the disclosed results, as appropriate, may, separately, or in any combination of such features, be utilised for realising the invention in diverse forms thereof.

While the invention has been described in conjunction with the exemplary embodiments described above, many equivalent modifications and variations will be apparent to those skilled in the art when given this disclosure. Accordingly, the exemplary embodiments of the invention set forth above are considered to be illustrative and not limiting. Various changes to the described embodiments may be made without departing from the spirit and scope of the invention.

For the avoidance of any doubt, any theoretical explanations provided herein are provided for the purposes of improving the understanding of a reader. The inventors do not wish to be bound by any of these theoretical explanations.

Any section headings used herein are for organizational purposes only and are not to be construed as limiting the subject matter described.

1. A computer-implemented method of predicting the effect of a drug combination, the method comprising:
obtaining, for a first drug and a second drug, the values of a plurality of features associated with the drug combination; and
predicting, using the values of said plurality of features, the effect of the drug combination using a machine learning model that has been trained to predict whether a drug combination is likely synergistic, using training data comprising for each of a plurality of training drug combinations: (i) the values of said plurality of features and (ii) an indication of whether the drug combination is synergistic;
wherein the plurality of features comprises at least one feature that characterises the similarity between one or more indications or diseases associated with one or more targets of the first drug and one or more indications or diseases associated with one or more targets of the second drug.

2. The method of claim 1, wherein the machine learning model has been trained to classify drug combinations between a plurality of categories comprising a synergistic category and a non-synergistic category, and wherein predicting the effect of the drug combination comprises determining whether the drug combination is likely to be in the synergistic category.

3. The method of any preceding claim, wherein the plurality of features comprises:
at least one feature that characterises the similarity between one or more indications associated with one or more targets of the first drug and one or more indications associated with one or more targets of the second drug, wherein indications are provided as Medical Subject Headings terms associated with the one or more targets of the drugs; and/or
at least one feature that characterises the similarity between one or more diseases associated with one or more targets of the first drug and one or more diseases associated with one or more targets of the second drug, wherein diseases are provided as disease ontology terms associated with the one or more targets of the drugs.

4. The method of any preceding claim, wherein the similarity between one or more indications or diseases associated with one or more targets of the first drug and one or more indications or diseases associated with one or more targets of the second drug is obtained as a semantic similarity metric between indication or disease terms associated with the one or more targets of the first drug and indication or disease terms associated with the one or more targets of the second drug, or a summarised metric that aggregates a plurality of such semantic similarity metrics each corresponding to a pair of indication or disease terms comprising an indication or disease term associated with a target of the first drug and an indication or disease term associated with a target of the second drug.

5. The method of any preceding claim, wherein the plurality of features comprise: one or more drug similarity features, one or more target similarity features, and one or more indication similarity features, optionally wherein the one or more drug similarity features are selected from: a side effect similarity, a mode of action similarity and a molecular structure similarity.

6. The method of claim 5, wherein the one or more target similarity features are selected from: a pathway similarity, a disease association similarity that characterises the similarity between one or more diseases associated with one or more targets of the first drug and one or more indications or diseases associated with one or more targets of the second drug, and a metric of closeness of the targets of the first and second drugs on a protein-protein interaction network.

7. The method of any of claim 5 or 6, wherein the one or more indication similarity features are selected from: an indication similarity metrics based on the target(s) of the drugs that characterises the similarity between one or more indications associated with one or more targets of the first drug and one or more indications associated with one or more targets of the second drug, and an indication similarity metric based on the drugs that characterises the similarity between indication annotations associated with the first and second drugs.

8. The method of claim 7, wherein the indication annotations are provided as anatomical therapeutic chemical (ATC) codes and/or wherein the indication similarity metric based on the drugs is selected from: a similarity metric that quantifies the similarity between multiple levels of the ATC codes for the first and second drugs, and a similarity metric that compares the first level of the ATC codes for the first and second drugs.

9. The method of claim 7 or claim 8, wherein a similarity metric that compares the first level of the ATC codes for the first and second drugs is a binary feature that indicates whether the first and second drugs are associated with ATC codes in the same first level category, or in a particular pair of ATC categories, and/or wherein the plurality of features comprises a set of binary features that indicate whether the first and second drugs are in each of a plurality of pairs of first level ATC categories, optionally wherein the plurality of pairs of first level ATC categories include LL, NN, JJ and CC.

10. The method of any preceding claim, wherein the machine learning model is selected from: decision trees and variants thereof including regularised and/or gradient boosted decision trees and random forest models, regularised discriminant analysis, logistic regression models, artificial neural networks, naïve Bayes classifiers, support vector machines and multivariate adaptive regression splines, and/or wherein the machine learning model is a non-linear model, and/or wherein the machine learning model is an ensemble model, and/or wherein the machine learning model is a tree-based model.

11. The method of any preceding claim, wherein the plurality of features comprises:
at least one feature that characterises the similarity between one or more indications associated with one or more targets of the first drug and one or more indications associated with one or more targets of the second drug, wherein indications are provided as Medical Subject Headings terms associated with the one or more targets of the drugs;
a side effect similarity,
a mode of action similarity,
a molecular structure similarity,
a pathway similarity,
a disease association similarity that characterises the similarity between one or more diseases associated with one or more targets of the first drug and one or more indications or diseases associated with one or more targets of the second drug, and a metric of closeness of the targets of the first and second drugs on a protein-protein interaction network, and
an indication similarity metric based on the drugs that characterises the similarity between indication annotations associated with the first and second drugs, wherein the indication annotations are provided as anatomical therapeutic chemical (ATC) codes and wherein the indication similarity metric based on the drugs comprises a similarity metric that quantifies the similarity between multiple levels of the ATC codes for the first and second drugs, and optionally a similarity metric that compares the first level of the ATC codes for the first and second drugs, wherein the similarity metric that compares the first level of the ATC codes for the first and second drugs is a set of binary features that indicate whether the first and second drugs are in each of a plurality of pairs of first level ATC categories, optionally wherein the plurality of pairs of first level ATC categories include LL, NN, and optionally one or more of JJ, CC, JS, DR, DS, CJ, AA, AM, AN, and BN.

12. The method of any preceding claim, wherein the machine learning model has been trained using a training data set comprising at least 1000, 2000 or 3000 unique drug combinations, and/or wherein the trained machine learning model has a specificity of at least 90% and sensitivity of 70% in predicting synergistic combinations when evaluated on unseen data, and/or wherein the method further comprises prior to providing the values said plurality of features as inputs to said machine learning model, applying one or more steps selected from: missing values imputation, filtering, standardisation and normalisation.

13. A method of providing a tool for predicting the effect of a drug combination, the method comprising:
obtaining, for each of a plurality of training drug combinations comprising a first drug and a second drug: (i) the values of a plurality of features associated with the drug combination; and (ii) information indicative of whether the drug combination is synergistic; and
training a machine learning model to predict whether a drug combination is likely synergistic, using said training data, wherein the machine learning model is trained to take as input the values of said plurality of features for a drug combination and to produce as output an indication of whether the rug combination is likely to be synergistic;
wherein the plurality of features comprise at least one feature that characterises the similarity between one or more indications or diseases associated with one or more targets of the first drug and one or more indications or diseases associated with one or more targets of the second drug.

14. A method of identifying a synergistic drug combination, the method comprising:
obtaining a plurality of candidate drug combinations;
predicting the effect of each of the plurality of candidate drug combinations using the method of any of claims 1 to 13; and
selecting a subset of the plurality of candidate drug combinations for experimental testing using the results of the predicting; and optionally further comprising testing the selected subset of the plurality of candidate drug combinations using one or more in vitro assays.

15. A system comprising:
at least one processor; and
at least one non-transitory computer readable medium containing instructions that, when executed by the at least one processor, cause the at least one processor to perform the method of any of claims 1 to 13.
